(19) United States
(12) Patent Application Publication
(10) Pub. No.: US 2022/0064111 A1
(43) Pub. Date: Mar. 3, 2022

(54) ENANTIOSELECTIVE SYNTHESIS OF BRIVARACETAM AND INTERMEDIATES THEREOF

(71) Applicant: CLININVENT RESEARCH PVT. LTD., Kolkata (IN)

(72) Inventors: Sarabindu Roy, 24-Parganas (South) (IN); Angshuman Ghosh, Jhargram (IN); Ramesh Dhondi Kubeer, Nizamabad (IN); Mutyala V. V. Vara Prasad, Mamidikudum Mandal (IN); Gopal Paul, Dist-Uttar Dinajpur (IN); Abhishek Garai, Dist-Paschim Medinipur (IN); Sourav Chakraborty, Dist-Paschim Medinipur (IN); Animesh Haldar, Kolkata (IN); Ajay Kumar Yadaw, Kolkata (IN); Subho Roy, Kolkata (IN)

(73) Assignee: CLININVENT RESEARCH PVT. LTD., Kolkata (IN)

(21) Appl. No.: 17/423,760

(22) PCT Filed: Jan. 17, 2020
(86) PCT No.: PCT/IN2020/050052

§ 371 Date: Jul. 16, 2021

(30) Foreign Application Priority Data
Jan. 17, 2019 (IN) 201931002041

(51) Int. Cl. C07D 207/27

(52) U.S. Cl. CPC C07D 207/27

(57) ABSTRACT

The present invention relates to an improved and economical process for enantioselective synthesis and purification of a novel key intermediate of Brivaracetam. Further, the present invention also relates to a process for the preparation of a chirally pure Brivaracetam of formula I utilizing the said intermediate.

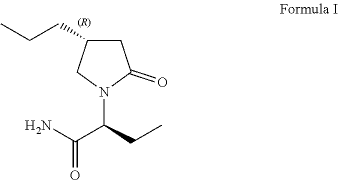

### FIELD OF INVENTION

The present invention relates to the field of process development chemistry. Particularly, the present invention relates to an improved and economical process for enantioselective synthesis and purification of a key intermediate of Brivaracetam. Further, the present invention also relates to a process for the preparation of Brivaracetam using this key intermediate.

### BACKGROUND OF THE INVENTION AND PRIOR ART

Brivaracetam is chemically known as (2S)-2-[(4R)-2-oxo-4-propyltetrahydro-1H-pyrrol-1-yl] butanamide, having the chemical structure of formula 1 as below:

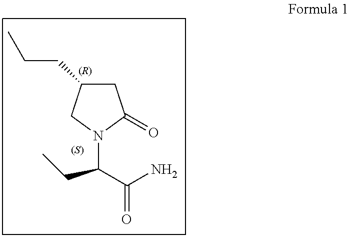

Brivaracetam is basically a chemical analogue of Levetiracetam, marketed under the brand name of BRIVIACT for the treatment as adjunctive therapy in the treatment of partial-onset seizures in patients at 16 years of age and older with epilepsy. Brivaracetam has an advantage over Levetiracetam in that it gets into the brain “much more quickly,” which means that “it could be used for status epilepticus, or acute seizures than cluster, or prolonged seizures”. From the Phase III trials, the self-reported rate of irritability with Brivaracetam was 2% for both drug doses (100 mg and 200 mg) Vs 1% for placebo, which compares to as much as 10% for levetiracetam in some post-marketing studies.

With the improved safety profile and possibility to be used for wider range of epilepsy, Brivaracetam is considered as one of the most promising 3^{rd} generation antiepileptic drugs.

Brivaracetam molecule is first disclosed in patent publication WO200162726, which describes 2-oxo-1-pyrrolidine derivatives and methods for their preparation. This patent publication further discloses compound (2S)-2-[(4R)-2-oxo-4-propyl-pyrrolidin-1-yl] butanamide which is known under the international non propriety name as Brivaracetam. As per Biopharmaceutics Classification System, Brivaracetam is a class I drug (High solubility and permeability).

Further, prior arts U.S. Pat. Nos. 6,784,197 and 7,629,474 disclose a process for synthesizing a diastereomeric mixture of (2S)-2-[(4R)-2-oxo-4-propylpyrrolidin-1-yl]-butanamide and (2S)-2-[(4S)-2-oxo-4-propylpyrrolidin-1-yl]-butanamide (Brivaracetam) which is purified by chiral HPLC (Scheme-I & Scheme-II respectively, as provided below). This process used for chiral resolution makes it difficult for bulk manufacturing as well as it affects the overall yield making the process uneconomical.

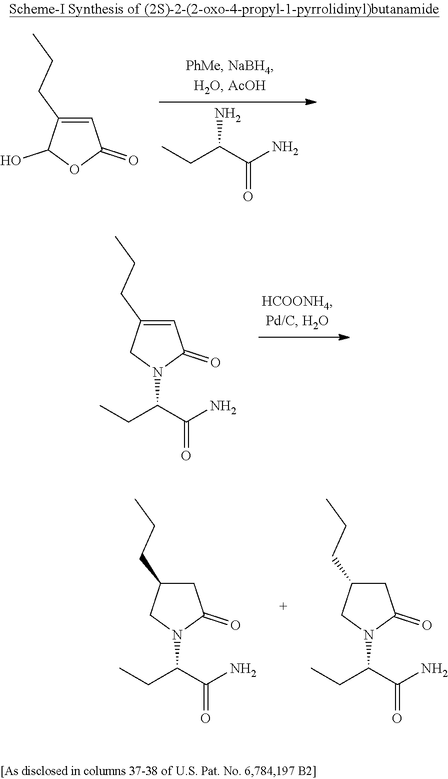

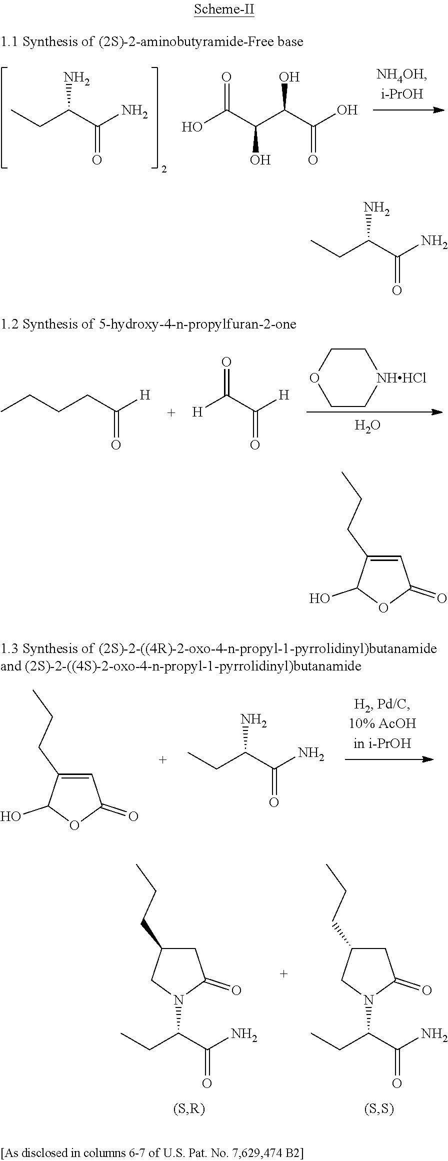

Moreover, some prior arts such as U.S. Pat. Nos. 7,122,682B2, 8,076,493B2, 8,338,621B2 and 8,957,226B2 also describe processes for preparing Brivaracetam, wherein, the purifications are reportedly done by chiral HPLC methods resulting into similar shortcomings.

Kenda et al.: Journal of Medicinal Chemistry, 2004, 47, 530-549 further proposes selection of (2S)-2-[(4R)-2-oxo-4-propylpyrrolidin-1-yl]butanamide 83α (ucb 34714; Brivaracetam) as the most interesting candidate showing 10 times more potency than Levetiracetam as an antiseizure agent in audiogenic seizure-prone mice. This article further discloses methods for synthesizing the said compound Brivaracetam. However, here too these compounds are synthesized as mixtures of stereoisomers (racemic or diastereoisomeric mixtures), separated by preparative HPLC on silica gel and/or chiral phases.

All these processes for the preparation of Brivaracetam as described in the above mentioned prior arts suffer from many disadvantages which includes difficulty in achieving desired chiral purity, tedious and cumbersome work up procedures, high temperature and longer reaction time, multiple crystallizations or isolation steps, use of excess reagents and solvents, column chromatographic separations & purifications etc. All these disadvantages affect the overall yield as well as the quality of the final product Brivaracetam and intermediates produced thereof; further, rendering such processes to be uneconomical and unsuitable for industrial scale-ups.

As a result, enantioselective synthesis of Brivaracetam and/or its key intermediates such as 4-Propyldihydrofuran-2(3H)-one was perceived to be a possible way of overcoming such problems in view of the large-scale synthesis. However, very few prior arts have been found to report successful reduction of such concept into practice.

Hughes et. al.: J. Am. Chem. Soc. 2003, 125 (37), p 11253-11258 discloses a dramatic acceleration of the enantioselective copper-catalyzed conjugate reduction of α,β-unsaturated lactones, upon addition of alcohol additives (shown below in Scheme III). Good to excellent yields and enantioselectivities are realized using a catalyst generated in situ from CuCl₂.H₂O, t-BuONa, p-tol-BINAP and PMHS (polymethylhydrosiloxane); however, this methodology is reportedly applied to the synthesis of (−)-Paroxetine, and not for Brivaracetam. Moreover, use of large amount of catalysts (5 mol %) and chiral ligands (5 mol %) such as (S)-p-tol-BINAP in this reported process along with extreme reaction parameters (e.g. maintaining cryogenic reaction conditions) renders this process expensive and industrially unsuitable.

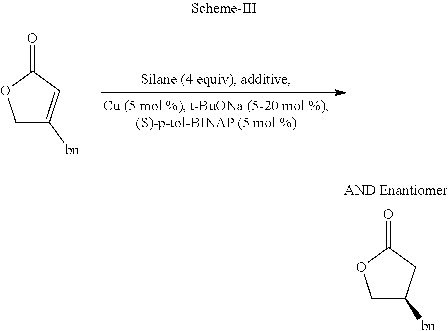

Furthermore, WO2016191435A1 relates to a process for a scalable synthesis of enantiomerically pure Brivaracetam, and related derivatives. It discloses a process for synthesis of the key intermediate of Brivaracetam, (4R)-4-Propyldihydrofuran-2(3H)-one which requires (R)-(−)-Epichlorohydrin and dialkyl malonate ester as the starting materials (as provided in scheme IV below). In the subsequent step, it involves Grignard reaction (with ethylmagnesium bromide) at low temperature (−30° C.) in presence of copper (I) iodide catalyst. In order to get the key intermediate, (4R)-4-propyldihydrofuran-2(3H)-one decarboxylation of alkyl (4R)-2-oxo-4-propyltetrahydrofuran-3-carboxylate is needed which disadvantageously requires high temperature (˜200° C.) and a high boiling polar solvent (e.g. DMSO, DMF, NMP).

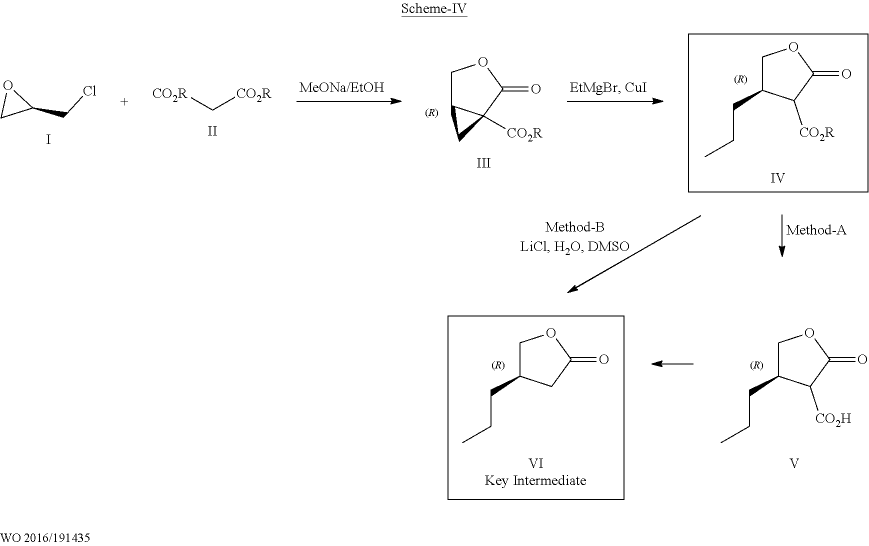

Further, among some randomly reported processes, WO2002070540A2 (on pages 12-13) discusses treating a lactone of formula (18) with a chiral amine such as (S)-methylphenylamine, in presence of a catalyst such as 2-hydroxypyridine, in a suitable solvent such as toluene, at reflux to afford a mixture of two diastereomers of Formula (22) and (23) which can be separated by silica gel flash chromatography. Such diastereomers when further reacted with an acid in dioxane affords a chiral lactone of Formula (26) or Formula (27).

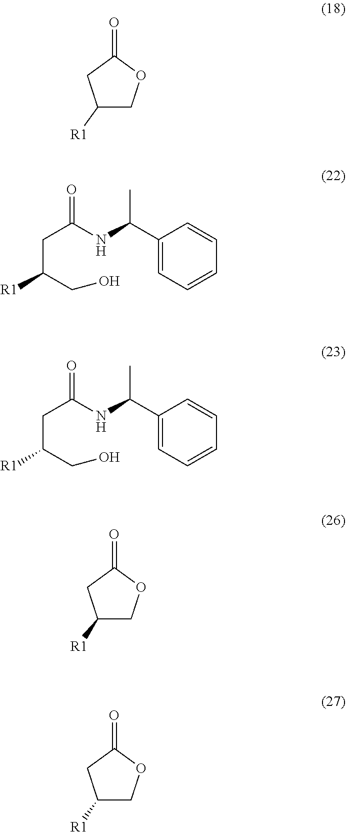

However, this prior reported process does not lead to synthesis of Brivaracetam as the target compound. Moreover, nothing is told about the chiral purity of the lactone produced by this process and the separation of the diastereomers involves conventional silica gel flash column chromatographic techniques, not suitable for practical industrial production.

Some bio-catalytic routes for preparing optically pure Brivaracetam and/or its key intermediates have also been reported, such as, Arnaud Schülé et. al.: Org. Process Res. Dev, 2016, 20(9), 1566-1575, wherein, the stereochemistry of the 4-n-propyl substituent is introduced by a bio-catalytic resolution of (rac)-methyl 2-propylsuccinate 4-tert-butyl ester. The chiral intermediate (R)-2-propylsuccinic acid 4-tert-butyl ester was obtained in only 42% yield and 97% ee. The biochemical method requires costly enzyme catalyst and also the control of pH, which is not suitable for industrial production.

Also, a recent publication—Ciceri et. al.: Molecules, 2018, 23, 2206-2216 discloses preparation of the optically pure key intermediate (R)-lactone of Brivaracetam by means of an enzymatic resolution under the classical irreversible trans-esterification conditions, of a suitable alcohol easily prepared; but the enantiopure alcohol obtained by Pseudomonas fluorescens lipase (PFL)-catalyzed transesterification requires longer reaction pathway and uses toxic, costly Ruthenium catalyst (RuCl₃) and sodium periodate in the subsequent oxidation step to reach the desired (R)-Lactone (i.e., (4R)-4-Propyldihydrofuran-2(3H)-one)-which again is not suitable for practical industrial production.

However, these bio-catalytic processes resulted mostly into lower yields of Brivaracetam and/or its key intermediates. Additionally, in such cases purifications were reportedly done by chromatographic techniques with their own demerits as discussed above.

Furthermore, a recently published prior art WO2018042393 (as shown in scheme-V below) discloses the enantioselective preparation of Brivaracetam using costly chiral auxiliary, S-4-phenyloxazolidine-2-one and valeryl chloride, followed by alkylation with tert-butylbromoacetate using LIHMDS as base at lower temperature (−55 to −60° C.) to produce the enantiomerically pure isomer (compound-V). Further hydrolysis of compound V, followed by reduction of a produced acid with BH₃.DMS and subsequent cyclization of the ester-alcohol intermediate with TFA furnishes the (R)-dihydro-4-propylfuran-2(3H)-one (compound-XI), the Brivaracetam intermediate (Scheme V).

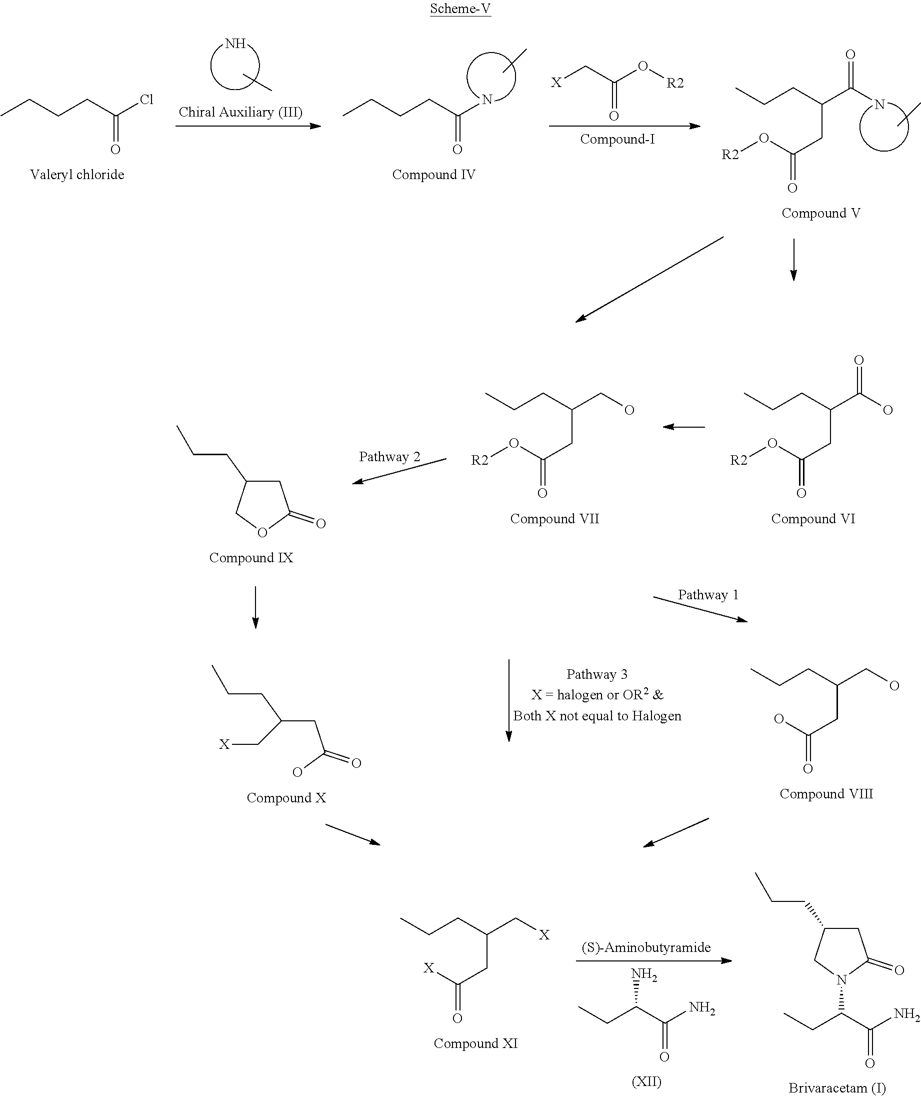

However, the process reported in this prior art involves using costly reagents along with extreme reaction parameters (e.g. cryogenic conditions) rendering it to be highly expensive and industrially unsuitable.

Accordingly, there is still a need in the art for an economical and improved process for the enantioselective synthesis of Brivaracetam and/or its intermediates with high purity and yield which overcomes the above drawbacks of prior arts.

Therefore, the present inventors have developed a novel, cost effective and efficient enantioselective process for the preparation of Brivaracetam and intermediates thereof which essentially avoids chiral resolutions of the racemic mixtures by chromatography. This currently developed process thus not only overcomes the drawbacks associated with the prior reported ones, but at the same time is industrially scalable, and produces a highly pure Brivaracetam with superior yields.

### OBJECTS OF THE INVENTION

An object of the invention is to overcome the disadvantages of the prior art.

Another object of the present invention is to provide a novel process for the preparation of enantiomerically pure Brivaracetam and intermediates thereof without involving any chiral chromatographic resolution technique.

Another object of the present invention is to provide a novel chirally pure diastereomeric intermediate 5 with 99.90-100% chiral purity:

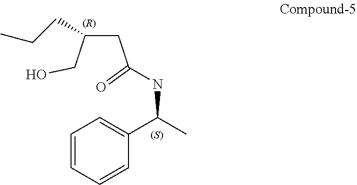

Yet another object of the present invention is to provide a new, economical process for preparing and chirally purifying the said diastereomeric intermediate 5, which is the key intermediate for the asymmetric (R)-lactone synthesis.

Yet another object of the present invention is to provide a new economical process for preparing a key Brivaracetam intermediate 6 i.e. (R)-lactone with 99.90-100% enantiomeric purity, from the said novel intermediate 5:

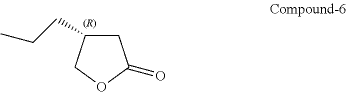

Still another object of the present invention is to provide a new, improved, economical process for synthesizing the chirally pure (R)-lactone intermediate 6 i.e. (4R)-4-Propyldihydrofuran-2(3H)-one, wherein the chiral ligand used is economical and at the same time its loading is as less as 0.1-1 mol %; and further, the said process is efficiently conducted at an ambient temperature range of 10° C. and 35° C. and within a short time of 5-10 hours.

A further object of the present invention is to provide a new, economical and industrially scalable process for the preparation of Brivaracetam i.e. (2S)-2-[(4R)-2-oxo-4-propylpyrrolidin-1-yl] butanamide from the said intermediate 5 or 6, with 99-100% chiral purity.

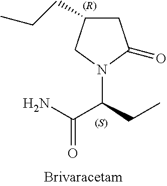

### SUMMARY OF THE INVENTION

One aspect of the present invention provides a new process for enantioselective synthesis of the compound of formula I (Brivaracetam) and key intermediates thereof comprising the steps of:

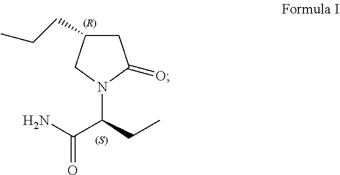

(a) condensing a pentanal with a glycoxylic acid in presence of a condensing agent to form Intermediate 1

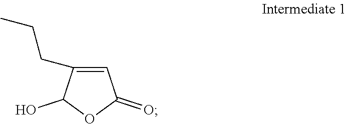

(b) reducing said Intermediate 1 with a reducing agent to form Intermediate 2

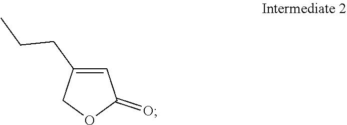

(c) treating said Intermediate 2 with 0.1-1 mol % of a chiral ligand in presence of a metal-based catalyst; followed by enantio-selectively reducing the unsaturated lactone of said Intermediate 2 in presence of a reductant and an additive in order to form Intermediate 3

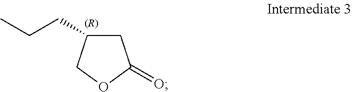

(d) reacting the said Intermediate 3 with a chiral amine in a solvent to produce a diastereomeric mixture of Intermediate 4

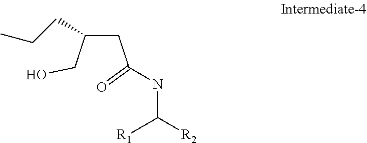

wherein R1 is a substituted or unsubstituted aryl or heteroaryl; and
R2 is a substituted or unsubstituted alkyl or cycloalkyl;

(e) purifying said Intermediate 4 by crystallization in order to form Intermediate 5 having 99.90-100% chiral purity

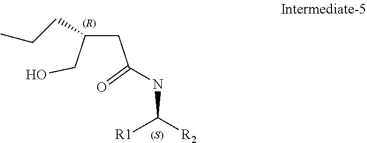

wherein R1 is a substituted or unsubstituted aryl or heteroaryl; and
R2 is a substituted or unsubstituted alkyl or cycloalkyl;

(f) cyclizing said Intermediate 5 with a cyclizing agent in order to form intermediate 6 having an enantiomeric purity of 99.90-100%

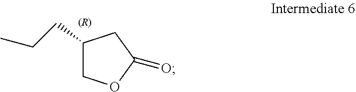

(g) reacting said intermediate 6 with a suitable ring-opening agent to produce the compound intermediate 7A

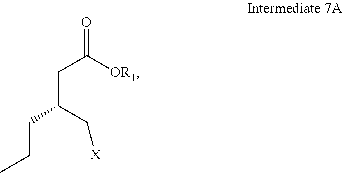

wherein R₁ is selected from saturated or unsaturated C₁₋₂₀ alkyl, substituted or unsubstituted C₁₋₁₀ aryl, a metal of Group I of the Periodic table; and
X is CI, Br, I, OH, OMs, OTs, ONs; with a proviso that X is OH only when R1 is a metal of Group I of the Periodic table;
OR
the compound intermediate 7B:

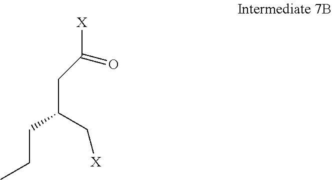

wherein X is selected from a group consisting of CI, Br, I, OMs, OTs, ONs; followed by

(h) reacting the said intermediate 7A OR intermediate 7B with a chiral amide to produce the compound Brivaracetam of formula I with chiral purity of 99-100%.

Another aspect of the present invention provides a new process for synthesizing Intermediate 3 from Intermediate 2 comprising steps of:

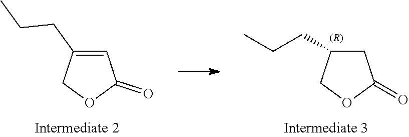

treating Intermediate 2 with a chiral ligand in a loading amount ranging between 0.1 and 1 mol %. in presence of a metal based catalyst; followed by

enantioselectively reducing the unsaturated lactone of said Intermediate 2 in presence of a reductant and an additive to form Intermediate 3.

Another aspect of the present invention provides a novel chirally pure diastereomeric intermediate 5 synthesized by the process as developed in the present invention:

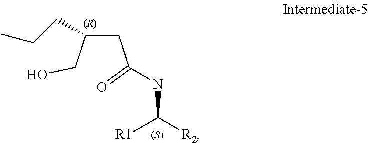

wherein R1 is substituted or unsubstituted aryl or heteroaryl; and R2 is substituted or unsubstituted alkyl or cycloalkyl; preferably

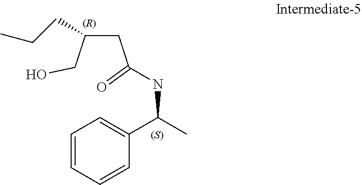

Yet another aspect of the present invention provides a new, economical process for chirally purifying the diastereomeric Intermediate 4 to form Intermediate 5 comprising step of:

crystallizing the said Intermediate 4 with a mixture of solvents in a volume range of 5:95 to 40:60 producing 99.90-100% chirally pure Intermediate 5.

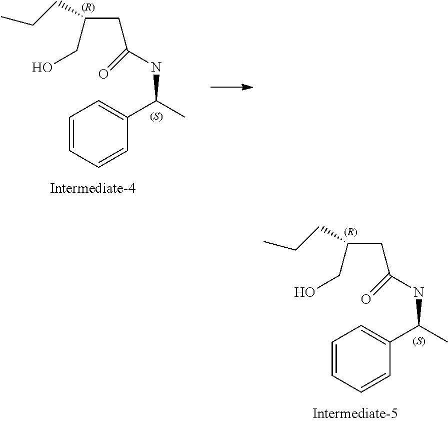

Yet another aspect of the present invention provides an enantiomerically pure Intermediate 6 with 99.90-100% enantiomeric excess synthesized by the process as developed in the present invention:

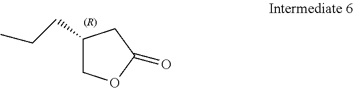

Yet another aspect of the present invention provides a chirally pure Intermediate 11 having formula:

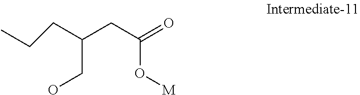

wherein, M is selected from a metal of Group I of the Periodic Table

A further aspect of the present invention provides a process for preparing a chirally pure key Intermediate 11, which comprises the step of reacting Intermediate 5 with a suitable base forming Intermediate 11:

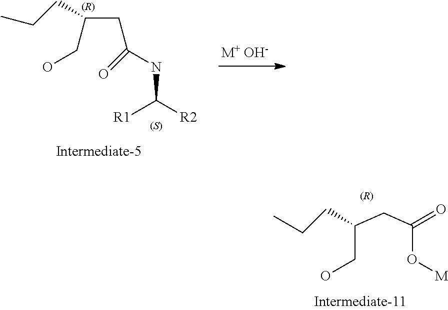

wherein R1 of Intermediate 5 is a substituted or unsubstituted aryl or heteroaryl; R2 of Intermediate 5 is a substituted or unsubstituted alkyl or cycloalkyl; and

M of Intermediate 11 is selected from a metal of Group I of the Periodic Table.

### BRIEF DESCRIPTION OF THE ACCOMPANYING DRAWINGS

FIGS. 1(A) and 1(B) graphically illustrate 1H NMR study results showing formation of Intermediate 1 [i.e. 5-hydroxy-4-propyl-5H-furan-2-one] in step-1 of the currently developed process of the present invention.

FIG. 2 graphically illustrates GC-MS (m/z) data having value of 141.0; confirming formation of Intermediate 1 of the present invention.

FIG. 3 graphically illustrates the proton NMR analysis data confirming formation of Intermediate 2 [i.e. 4-propylfuran-2(5H)-one] of the present invention.

FIG. 4 graphically illustrates the GC-MS (m/z) analysis data showing a value of 126.1; thus, confirming formation of Intermediate 2 of the present invention

FIG. 5 graphically illustrates the proton NMR data confirming formation of Intermediate 3 [i.e. (R)/(S)-4-propyldihydrofuran-2-one] of the present invention.

FIG. 6 graphically illustrates the GC-MS analysis data showing a value of (m/z)=128.1, confirming the formation of Intermediate 3 of the present invention.

FIGS. 7(A) and 7(B) graphically illustrate the Chiral GC analysis data of Intermediate 3 prepared from 0.1 mol % and 0.5 mol % of S-BINAP.

FIG. 8 graphically illustrates proton NMR analysis data that supports formation of Intermediate 4 of the present invention.

FIG. 9 graphically illustrates GC-MS analysis data showing a value of (m/z)=249.2, confirming the formation of Intermediate 4 of the present invention.

FIG. 10 graphically illustrates chiral HPLC analysis data confirming formation of Intermediate 4 i.e. (3R)-3-(hydroxymethyl)-N-[(1S)-1-phenylethyl]hexanamide with diastereomeric excess.

FIG. 11 graphically illustrates proton NMR analysis data that confirms the formation of Intermediate 5 of the present invention.

FIG. 12 graphically illustrates GC-MS analysis data showing a value of (m/z)=249.2, confirming the formation of Intermediate 5 of the present invention.

FIG. 13 graphically illustrates Chiral HPLC analysis data confirming the formation of Intermediate 5 i.e. (3R)-3-(hydroxymethyl)-N-[(1S)-1-phenylethyl] hexanamide with 100% diasteromeric excess.

FIG. 14 graphically illustrates proton NMR analysis data that confirms the formation of Intermediate 6 of the present invention.

FIG. 15 graphically illustrates GC-MS analysis data having value of (m/z)=128.1, confirming formation of Intermediate 6 of the present invention.

FIG. 16 graphically illustrates chiral GC analysis data showing an Enantiomeric ratio (R:S): 100:0, confirming the formation of (R)-4-propyldihydrofuran-2-one i.e. Intermediate 6 of the present invention.

FIG. 17 graphically illustrates the proton NMR analysis data that confirms the formation of Intermediate 7 of the present invention.

FIG. 18 graphically illustrates GC-MS analysis data having value of 207.1, confirming the formation of Intermediate 7 of the present invention with 100% purity.

FIG. 19 graphically illustrates the proton NMR analysis data that confirms the formation of Intermediate 8 of the present invention.

FIG. 20 graphically illustrates the GC-MS analysis data having values of 237.2 and 239.2, confirming formation of Intermediate 8 of the present invention.

FIG. 21 graphically illustrates NMR analysis data confirming formation of the final product Brivaracetam from Intermediate 8.

FIG. 22 graphically illustrates GC-MS (m/z) analysis data having value of 212.2; thus confirming formation of Brivaracetam of the present invention.

FIG. 23 graphically illustrates chiral HPLC analysis data confirming formation of Brivaracetam of the present invention with 100% diastereomeric excess.

FIG. 24 graphically illustrates NMR analysis data confirming formation of Intermediate 9 of the present invention.

FIG. 25 graphically illustrates NMR analysis data confirming formation of the final product Brivaracetam from Intermediate 9.

FIG. 26 graphically illustrates NMR analysis data confirming formation of Intermediate 10 of the present invention.

FIG. 27 graphically illustrates NMR analysis data confirming formation of the final product Brivaracetam from Intermediate 10.

FIG. 28 graphically illustrates NMR analysis data confirming formation of the Intermediate 11 from Intermediate 6.

FIG. 29 graphically illustrates NMR analysis confirming the formation of Intermediate 11 from Intermediate 5.

Persons skilled in the art will appreciate that elements in the figures are illustrated for simplicity and clarity and may have not been drawn to scale. For example, the dimensions of some of the elements in the figure may be exaggerated relative to other elements to help to improve understanding of various exemplary embodiments of the present disclosure. Throughout the drawings, it should be noted that like reference numbers are used to depict the same or similar elements, features, and structures.

### DETAILED DESCRIPTION OF THE INVENTION

The following description with reference to the accompanying drawings is provided to assist in a comprehensive understanding of exemplary embodiments of the invention. It includes various specific details to assist in that understanding but these are to be regarded as merely exemplary.

Accordingly, those of ordinary skill in the art will recognize that various changes and modifications of the embodiments described herein can be made without departing from the scope of the invention. In addition, descriptions of well-known functions and constructions are omitted for clarity and conciseness.

The terms and words used in the following description and claims are not limited to the bibliographical meanings, but, are merely used by the inventor to enable a clear and consistent understanding of the invention. Accordingly, it should be apparent to those skilled in the art that the following description of exemplary embodiments of the present invention are provided for illustration purpose only and not for the purpose of limiting the invention as defined by the appended claims and their equivalents.

It is to be understood that the singular forms “a,” “an,” and “the” include plural referents unless the context clearly dictates otherwise.

Features that are described and/or illustrated with respect to one embodiment may be used in the same way or in a similar way in one or more other embodiments and/or in combination with or instead of the features of the other embodiments.

It should be emphasized that the term “comprises/comprising” when used in this specification is taken to specify the presence of stated features, integers, steps or components but does not preclude the presence or addition of one or more other features, integers, steps, components or groups thereof.

The term “cost-efficient or economical” as used in the specification refers to the cost of synthesizing Brivaracetam and/or its key intermediates such as (4R)-4-Propyldihydrofuran-2(3H)-one, (3R)-3-(hydroxymethyl)-N-[(1S)-1-phenylethyl] hexanamide which involves lower loading of a cheaper chiral ligand and is essentially conducted at a comparatively lesser time and at ambient temperatures (comparative data as presented in the specification below); therefore, making it suitable for industrial scale-ups.

The term “improved process” as used in the specification refers to a process for the preparation of enantiomerically pure Brivaracetam and key intermediates thereof without involving any chiral chromatographic resolution technique.

The term “cheaper chiral ligand” as used in the specification refers to use of (S)-BINAP [(2,2′-bis(diphenylphosphino)-1,1′-binaphthyl)] in the currently developed process which is comparatively less expensive with respect to other chiral ligands as reportedly used in the prior arts (comparative data as presented in the specification below).

The term “lower chiral ligand loading” as used in the specification refers to use of lower amount of chiral ligand/reagent in the currently developed process than those reported in the prior arts (comparative data as presented in the specification below).

The term “chirally pure” as used in the specification refers to synthesizing Brivaracetam and/or its key intermediates with 99-100% enantiomeric purity.

The present invention relates to a new, improved, economical process for the preparation of enantiomerically pure Brivaracetam and/or its key intermediates thereof without involving any chiral chromatographic resolution technique.

Such preparation method of Brivaracetam and intermediate compounds thereof as obtained during the process of the present invention is described below. The currently developed process is embodied in many different forms and should not be construed as being limited to the description set forth herein.

An embodiment of the present invention provides a new, cost effective and easily scalable process for the preparation of Brivaracetam i.e. (2S)-2-[(4R)-2-oxo-4-propylpyrrolidin-1-yl] butanamide, with 99-100% chiral purity:

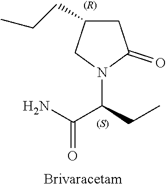

In a further specific embodiment, the reaction conditions for each reaction step of the present invention are detailed below:

Step 1: a pentanal and glyoxylic acid are made to condense, essentially in presence of morpholine to form 5-hydroxy-4-propyl-5H-furan-2-one (compound/intermediate 1):

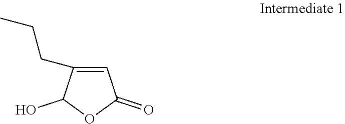

Step 2: the said compound 1 is later treated with a reducing agent selected from sodium borohydride or lithium borohydride in order to afford the desired lactone i.e. 4-propylfuran-2(5H)-one (compound/intermediate 2):

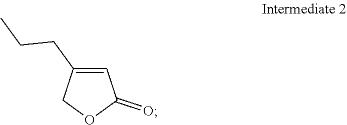

next

Step 3: (a) the desired stereochemistry of the 4-n-propyl substituent is introduced into the said compound 2 by means of adding a metal salt, preferably a copper (Cu)-salt as catalyst [for e.g., CuI, CuCl, CuCl₂, Cu(OAc)₂, CuO, Cu(NO₃)₂, or CuBr] and a chiral ligand selected from S-BINAP, S-tol-BINAP, S-BIPHEMP, or (R)-SEGPHOS, preferably 5-BINAP; and then sequentially

(b) enantioselective 1,4-reduction of the unsaturated lactone of said compound 2 is carried out in presence of a reductant selected from a group consisting of PMHS (polymethylhydrosiloxane), 1,1,3,3-Tetramethyldisiloxane, Et₃SiH (triethyl siliane) and Ph₂SiH₂ (diphenylsilane), along with an additive chosen from water, methanol, ethanol, propanol, pentanol, t-butanol, n-butanol, amyl alcohol, isopropyl alcohol and mixtures thereof;

wherein the desired (4R)-4-propyldihydrofuran-2(3H)-one (compound/intermediate 3) is obtained with 75-80% yield and 80-90% ee; further followed by

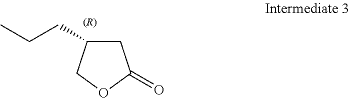

Step 4: treatment of the said enantiomerically rich (4R)-4-propyldihydrofuran-2(3H)-one i.e. compound 3 with a chiral amine selected from a group consisting of (S)-1-Phenylethylamine, (S)-1-bromophenylethylamine, (S)-1-methoxyphenylethylamine, (S)-1-tolylethylamine and (S)-(−)-1-(1-naphthyl)ethylamine, (R)-1-Phenylethylamine, (R)-1-bromophenylethylamine, (R)-1-methoxyphenylethylamine, (R)-1-tolylethylamine and (R)-(+)-1-(1-naphthyl)ethylamine in a solvent such as water, toluene, t-butanol, xylene and acetonitrile, isopropyl acetate, dichloromethane, ethyl acetate, cyclohexane and mixtures thereof in order to produce a diastereomeric mixture (compound/intermediate 4) (RS and SS) with 80-90% diastereomeric excess;

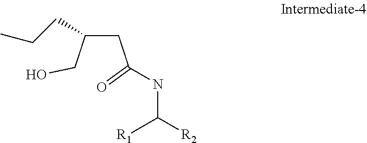

wherein R1 is a substituted or unsubstituted aryl or heteroaryl; and R2 is a substituted or unsubstituted alkyl or cycloalkyl;

Step 5: the said compound 4 is further separated as a novel, pure diastereomer (RS) (compound/intermediate 5) with 75-85% yield and 99.90-100% diastereomeric excess by preferential crystallization:

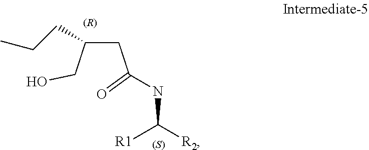

wherein R1 is substituted or unsubstituted aryl or heteroaryl; and R2 is substituted or unsubstituted alkyl or cycloalkyl; preferably

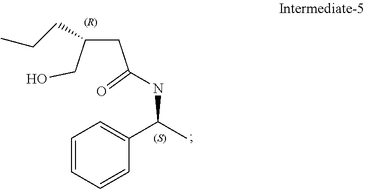

Step 6: cyclization of the desired diasteromer [(3R)-3-(hydroxymethyl)-N-[(1S)-1-phenylethyl] hexanamide] (compound/intermediate 5) in presence of cyclizing agent selected from dioxane-HCl, HCl, HI, H₂SO₄, HBr and so on and so forth, in order to produce the desired enantiomerically pure (4R)-4-propyldihydrofuran-2(3H)-one (compound/intermediate 6) with 90-95% yield and 99.90-100% ee:

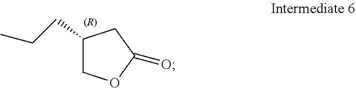

Steps 7 to 16: the said enantiomerically pure (R)-lactone i.e. compound 6 is thus used for enanatioselectively producing Brivaracetam i.e. (2S)-2-[(4R)-2-oxo-4-propyltetrahydro-1H-pyrrol-1-yl] butanamide via steps 7-10 or steps 11-13 or steps 14-16 or steps 17A/17B, as shown below in schemes B, C, D respectively. The said schemes B and C draw reference from some prior arts like Arnaud Schülé et. al.: Organic Process Research & Development, 2016, 20 (9), 1566-1575 and IN201717005820.

The metal based catalyst in the step of forming said Intermediate 3 is selected from CuI, CuCl, CuCl₂, Cu(OAc)₂, CuO, Cu(NO₃)₂ or CuBr and the chiral ligand in the step of forming said Intermediate 3 is selected from a group consisting of S-BINAP, S-tol-BINAP, S-BIPHEMP and (R)-SEGPHOS, preferably S-BINAP.

The reductant used in the step of forming said Intermediate 3 is selected from a group consisting of PMHS (polymethylhydrosiloxane), 1,1,3,3-Tetramethyldisiloxane, Et₃SiH (triethyl siliane) and Ph₂SiH₂ (diphenylsilane) and additive used for preparing Intermediate 3 is selected from water, methanol, ethanol, propanol, pentanol, t-butanol, n-butanol, amyl alcohol, isopropyl alcohol and mixtures thereof.

The reaction maintained is conducted at a temperature ranging between −10° C. and 40° C., preferably between 10° C. and 35° C. for the step of forming said Intermediate 3.

The chiral amine used in step (d) is selected from a group consisting of (S)-1-Phenylethylamine, (S)-1-bromophenylethylamine, (S)-1-methoxyphenylethylamine, (S)-1-tolylethylamine and (S)-(−)-1-(1-naphthyl)ethylamine, (R)-1-Phenylethylamine, (R)-1-bromophenylethylamine, (R)-1-methoxyphenylethylamine, (R)-1-tolylethylamine and (R)-(+)-1-(1-naphthyl)ethylamine and the solvent used in step (d) is selected from water, toluene, t-butanol, xylene and acetonitrile, isopropyl acetate, dichloromethane, ethyl acetate, cyclohexane and mixtures thereof.

The intermediate 4 is produced in step (d) with 80-90% diastereomeric excess.

The cyclizing agent in step (f) of the process is selected from HCl, HBr, HI, HNO₃, CH₃COCl, SOCl₂, TMsCl, H₂SO₄ or any Lewis acid and the ring-opening agent used in step (g) is selected from a group consisting of SOCl₂, ZnCl₂, acetic anhydride, acetic acid, LiOH, NaOH, KOH, HCl, HI and HBr.

The amide utilized in step (h) of the process is selected from (S)-2-aminobutanamide, alkyl-(S)-2-aminobutanoate and salts thereof.

The above process of the present invention is schematically represented in Scheme A below:

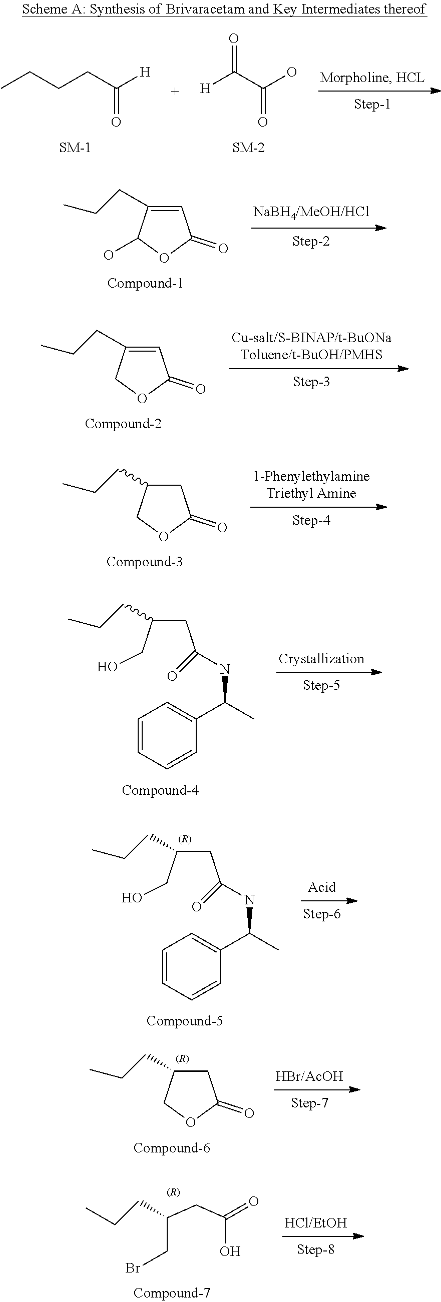

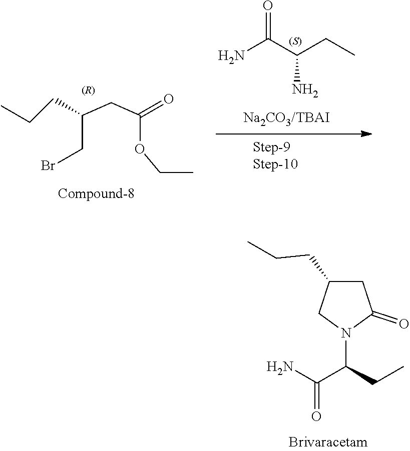

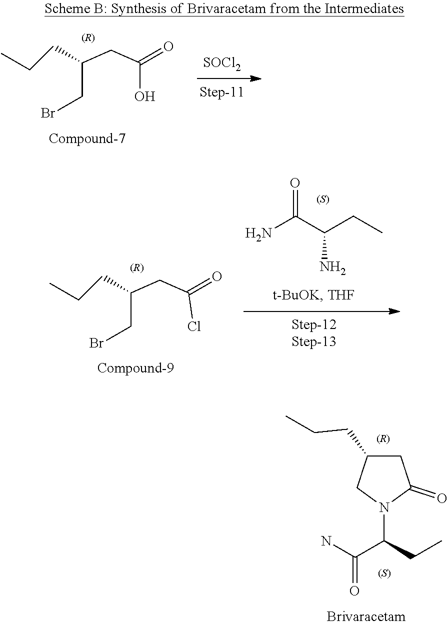

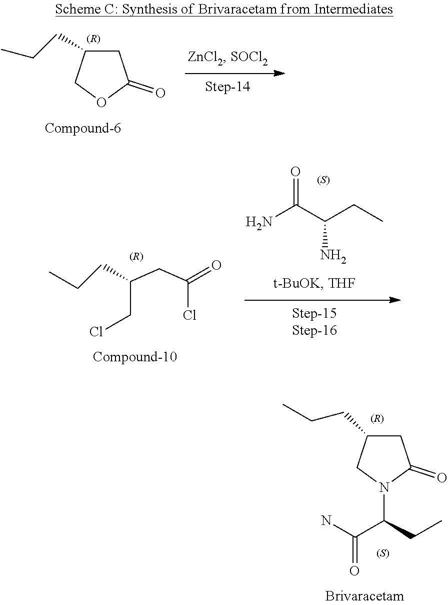

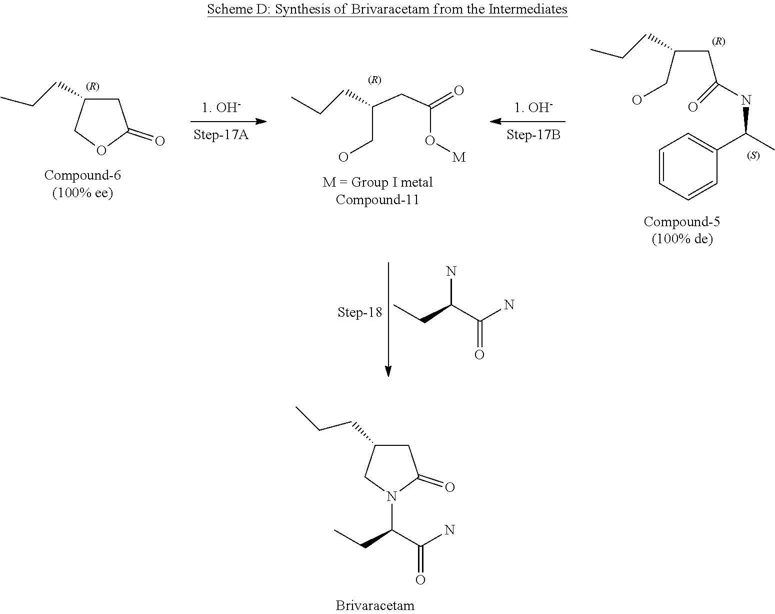

Accordingly, another important embodiment of the present invention is to synthesize a new, chirally pure diastereomeric compound/intermediate 5 with 99.90-100% chiral purity, which also serves as the key intermediate for synthesizing asymmetric (R)-lactone synthesis (compound/intermediate 6):

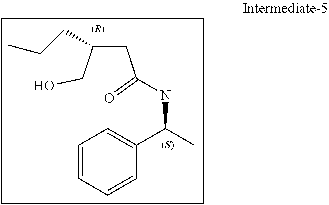

A further embodiment of the present invention is to provide a new, economical process for chirally purifying the diastereomeric intermediate 4 to form the novel intermediate 5 by crystallization technique:

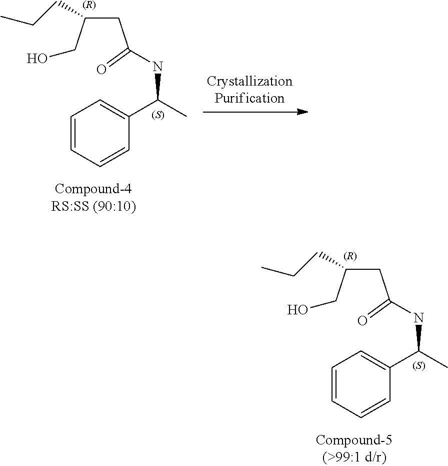

In accordance with the above method of the present invention, intermediate 4 is purified to form intermediate/compound 5 by means of crystallization technique, which is known to provide a higher extent of purification of mixtures than other conventionally known chromatographic methods. Accordingly, in the present invention Intermediate 4 is preferentially crystallized by means of reaction the same with a mixture of solvents selected from di-isopropyl ether, di-isopropyl acetate, diethyl ether, isopropyl acetate, methyl tertiary butyl ether, isopropyl acetate and mixtures thereof, in a volume range of 5:95 to 40:60, thus producing a pure enantioselective compound/intermediate 5 with 75-85% yield and 99.90-100% chiral purity. Such compound 5 then further acts as a key intermediate for producing (R)-lactone (compound/intermediate 6) with high purity, and eventually synthesizing pure Brivaracetam from the same.

Yet another embodiment of the present invention provides an enantiomerically rich (4R)-4-propyldihydrofuran-2(3H)-one [(R)-lactone] i.e. key Brivaracetam intermediate 6 with 99.90-100% enantiomeric purity:

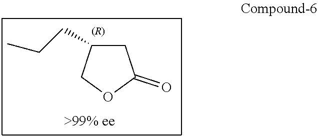

In the present invention, such chirally pure (R)-lactone compound/intermediate 6 i.e. (R) 4-Propyldihydrofuran-2(3H)-one is thus synthesized by a new, improved and economically viable process as described in steps 1-6 in scheme A above, wherein the crucial step 3 offers following advantages in view point of industrial scale usage:

the copper (Cu) salt catalyst and the chiral ligand used is selected from S-BINAP, S-tol-BINAP, S-BIPHEMP or (R)-SEGPHOS, preferably S-BINAP which is economical;

the said chiral ligand is essentially loaded at a lower amount ranging between 0.1 to 1.0 mol %;

the said process is efficiently conducted within a short time range of 5-10 hours; and at an ambient temperature range of −10 to 40° C., preferably at 10-35° C. without requiring any cryogenic conditions.

Furthermore, in accordance with the present invention, said Intermediate 6 is made to react with a suitable ring-opening agent selected from SOCl₂, ZnCl₂, acetic anhydride, acetic acid, LiOH, NaOH, KOH, HCl, HI, HBr and/or mixtures thereof, in order to produce the compounds falling within the scope of intermediate 7A

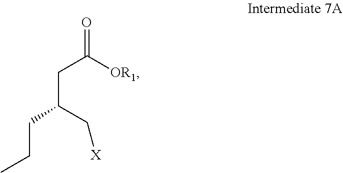

wherein R₁ is selected from saturated or unsaturated C₁₋₂₀ alkyl, substituted or unsubstituted C₁₋₁₀ aryl, a metal of Group I of the Periodic table; and

X is CI, Br, I, OH, OMs, OTs, ONs; with a proviso that X is OH only when R1 is a metal of Group I of the Periodic table

OR

the compound intermediate 7B:

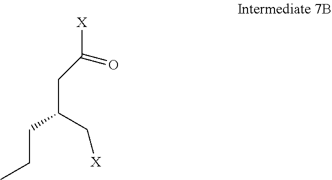

wherein X is selected from a group consisting of CI, Br, I, OMs, OTs, ONs;
such that said intermediate 7A OR intermediate 7B further undergoes chiral amidation to produce Brivaracetam of 99-100% chiral purity. The chiral amide used herein, is essentially selected from (S)-2-aminobutanamide, alkyl-(S)-2-aminobutanoate and/or salts thereof.

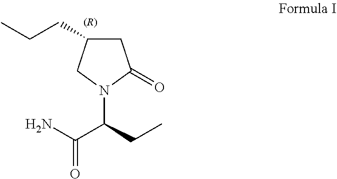

Yet another embodiment of the present invention provides a key Compound/Intermediate 11:

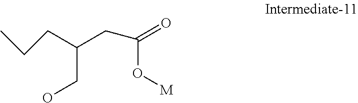

wherein, M is selected from a metal of Group I of the Periodic Table

In a further specific embodiment of the present invention Intermediate 11 is:

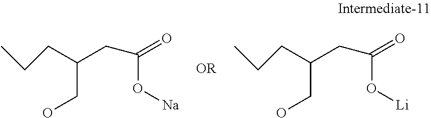

Another embodiment of the present invention provides a process for synthesizing the said Intermediate 11 from Intermediate 5, comprising the step of reacting Intermediate 5 with a suitable base forming Intermediate 11 (as mentioned above in scheme D):

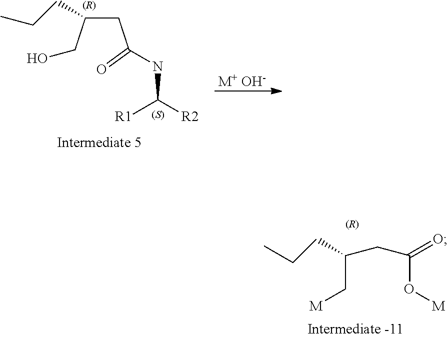

preferably

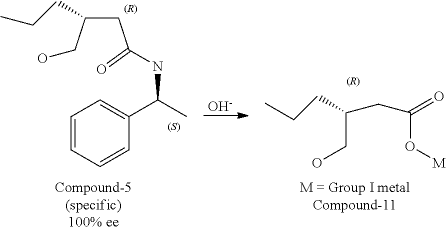

Further, another embodiment of the present invention provides a process for synthesizing the said Intermediate 11 from Intermediate 6 comprising the step of reacting Intermediate 6 with a suitable base forming Intermediate 11 (as mentioned above in scheme D):

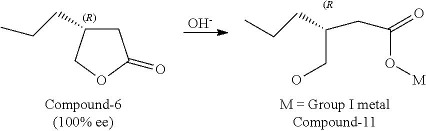

In accordance with the process of the present invention, the suitable base used in forming Intermediate 11 is chosen from NaOH, LiOH, KOH.

The comparative advantages of the present invention with respect to the closest prior arts are provided in the example section below.

The invention is now illustrated by way of non-limiting examples. The examples are intended to be purely exemplary of the invention, should therefore not be considered to limit the invention in any way.

### EXAMPLES

### Example 1: Synthesis of 5-hydroxy-4-propyl-5H-furan-2-one [Compound/Intermediate 1] (Step-1)

Example 1 illustrates the process for preparing 5-hydroxy-4-propyl-5H-furan-2-one (Intermediate 1) as done in the present invention.

### Procedure

In the first step of the present invention, the reactor is charged with heptane (4.04 L, 4.04 vol) and morpholine (1.17 L, 14 mol, 1.09 eq). The solution is then stirred at approximately 22° C. for 10 min, before being cooled to 4.4° C. Further, a 50% aqueous solution of glyoxylic acid (1000 g, 13.5 mol, 1.0 eq.) is slowly added to the above solution, while maintaining the temperature below 40° C. The reaction medium is then stirred for 2 hours at a temperature between 23.8° C. and 30.9° C. Furthermore, valeraldehyde (pentanal) (1.52 L, 14.3 mol, 1.06 eq) is slowly added to the said reaction medium, while maintaining the temperature below 40° C. After addition of valeraldehyde, the reaction mixture is heated between 40.1° C. and 41.7° C. for 18.04 hours.

The reaction mixture is then cooled to 22.8° C. and an aqueous solution of hydrochloric acid (1.25 L, 1.73 eq) is added to it, while keeping the temperature between 23.5 and 25.0° C. followed by stirring for 4 hours.

The reaction mixture is then allowed to separate organic phase from aqueous phase. The aqueous phase is washed thrice with heptane (2 L, 2 vol) followed by three times extraction with dichloromethane (3 L, 3 vol) to form Compound/Intermediate-1. The combined organic phase is then washed with a 20 % w/w aqueous solution of sodium chloride (1.6 L, 1.6 Vol). Further, the organic layer is dried by azeotropic distillation under vacuum at a jacket temperature of maximum 40° C. and then filtered. Finally, the reaction mixture is concentrated under vacuum, below 40° C. to obtain said Intermediate-1 (1.75 kg, 12.3 mol, 91.1% Yield). Analytical characterization is carried on formed Intermediate 1 to further confirm the formation of the same with desired attributes.

### Results

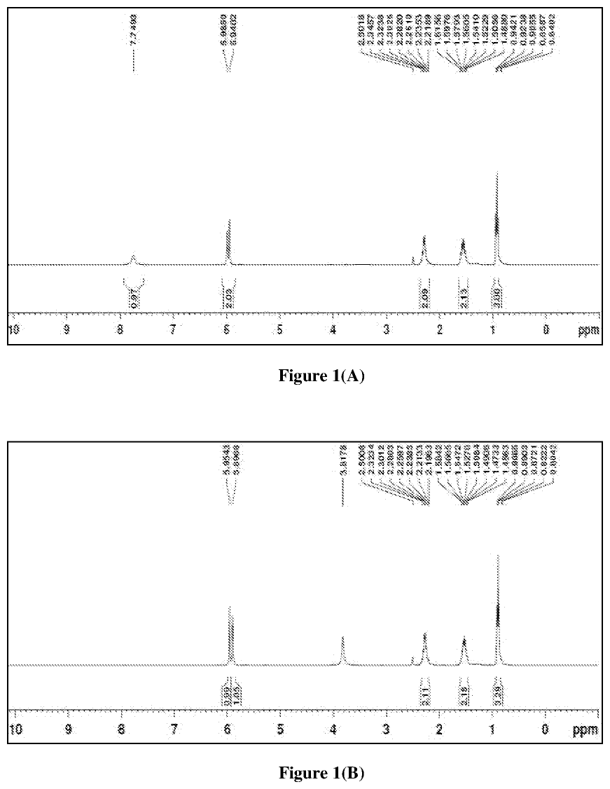

i) The proton NMR analysis data as illustrated in accompanying FIG. 1(A) depicts the following data: 1H NMR (400 MHz, DMSO-d6): δ 0.93 (t, J=7.4 Hz, 3H), 1.47-1.63 (m, 2H), 2.22-2.36 (m, 2H), 5.95 (s, 1H), 5.98 (s, 1H), 7.75 (br s, 1H, exchange with D₂O [FIG. 1(B)]; confirming formation of Intermediate 1 of the present invention.

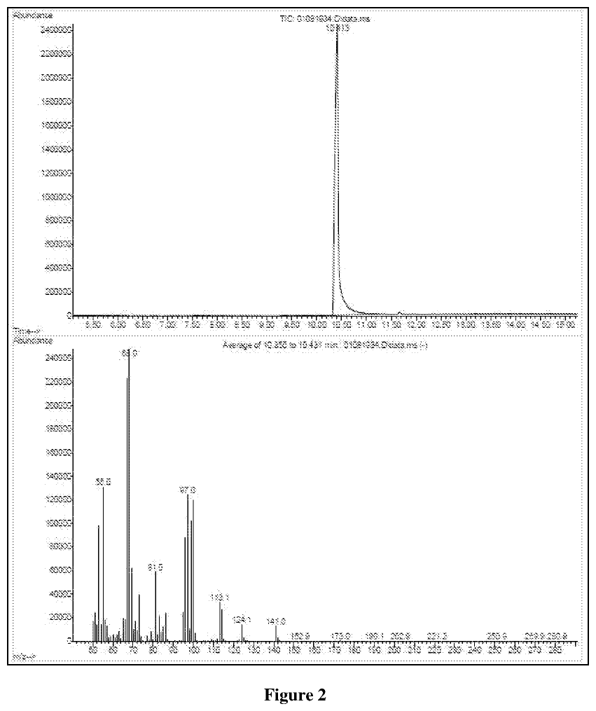

ii) The GC-MS (m/z) analysis as illustrated in accompanying FIG. 2 provides a value of 141.0; confirming formation of Intermediate 1 of the present invention.

### Example 2: Synthesis of 4-propyl-5H-furan-2-one [Compound/Intermediate 2] (Step-2)

Example 2 illustrates the process for preparing 4-propyl-5H-furan-2-one (Compound/Intermediate 2) from Intermediate 1 of example 1 above by the process as developed in the present invention.

### Procedure

In step 2 of the present invention, Sodium borohydride (83.1 g, 2.2 mol) is added in portions to Intermediate-1 (250 g, 1.75 mol) of example 1 taken in methanol (2000 mL), followed by stirring at room temperature for 1 h. Further, the reaction mixture is concentrated and 2 M HCl (1.25 L) is added to it. The reaction mixture is allowed for phase separation. Furthermore, the aqueous phase is extracted with dichloromethane (1.5 L) and the organic phase is dried over sodium sulphate (Na₂SO₄) and subsequently concentrated to give Compound/Intermediate-2 (200 gm, 90.15% yield). Analytical characterization is carried on formed Intermediate 2 to further confirm the formation of the same with desired attributes.

### Results

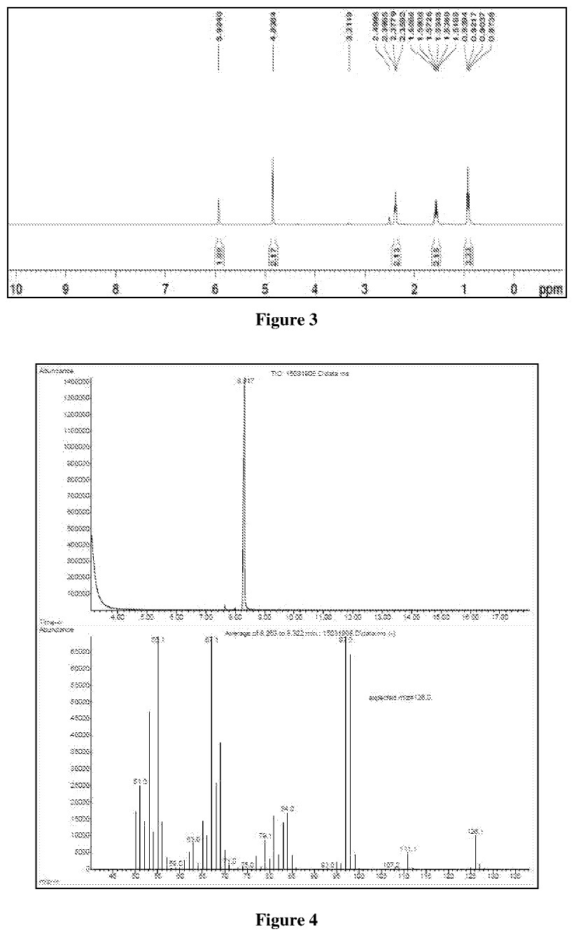

i) The proton NMR analysis data as illustrated in accompanying FIG. 3 depicts the following data: ¹H NMR (400 MHz, DMSO-d₆): δ 0.92 (t, J=7.36 Hz, 3H), 1.56 (sextet, J=7.48 Hz, 2H), 2.37 (t, J=7.48 Hz, 2H), 4.84 (s, 2H), 5.92 (s, 1H), that further supports formation of Intermediate 2 of the present invention.

ii) The GC-MS (m/z) analysis as illustrated in accompanying FIG. 4 provides a value of 126.1; confirming formation of Intermediate 2 of the present invention.

### Example 3: Synthesis of (R)/(S)-4-propyldihydrofuran-2-one [Compound/Intermediate 3] (Step-3)

Example 3 illustrates the process for preparing (R)/(S)-4-propyldihydrofuran-2-one (Compound/Intermediate 3) from Intermediate 2 of example 2 above by the process as developed in the present invention.

### Procedure

### Example 3(A)

In step 3(A) of the present invention, Copper(I) chloride (0.786 g, 7.937 mmole), Sodium tert-butoxide (4.576 g, 47.619 mmole), S-BINAP (2.47 g, 3.968 mmole) are taken in N₂ purged Toluene (7 L) in a round bottom flask. The reaction mass is then stirred for 1 hr at a temperature between 25 to 30° C. Further, polymethylhydrosiloxane (PMHS) (673.854 mL, 11904.762 mole) is added to the reaction mass and stirred for 2 hrs. Intermediate 2 of the present invention i.e. 4-propylfuran-2(5H)-one (500 g, 3968.254 mmole) is dissolved in a mixture of toluene (500 mL), water (in catalytic amount) and tertiarybutanol (1.5 L), which is added slowly to the reaction mass under N₂ at 0° C. Temperature of the reaction mass is increased slowly to 25-30° C. with constant stirring. After completion of the reaction, the reaction mass is quenched with NaOH solution, filtered and subsequently acidified with aq. HCl. The reaction mass is further extracted with dichloromethane to obtain crude (4R)-4-propyldihydrofuran-2(3H)-one, which is purified by vacuum distillation to get pure (4R)-4-propyldihydrofuran-2(3H)-one i.e. Intermediate 3 (400 g, 78.65% yield, 82.02% ee). Analytical characterization is carried on formed Intermediate 3A to further confirm the formation of the same with desired attributes.

### Example 3(B)

In step 3(B) of the present invention, CuCl (3.928 g, 39.682 mmole), tBuONa (4.576 g, 47.619 mmole), S-BINAP (12.345 g, 19.841 mmole) are added to N₂ purged toluene (7 L), taken in a round bottom flask, and stirred for 1 hr at 25° C. Further, PMHS (673.854 mL, 11904.762 mole) is added to the reaction mixture and stirred for 2 hrs. Furthermore, 4-propylfuran-2(5H)-one (500 g, 3968.254 mmole) is dissolved in toluene (500 mL), and water (in catalytic amount) to which tertiary butanol (1.5 L) is added slowly under N₂ at 0° C. The Reaction mass is put under stirring while allowing the temperature to rise slowly between 25-30° C. After completion of reaction, the reaction mass is quenched with NaOH solution, and subsequently filtered and acidified with aq. HCl. Further, the acidified reaction mixture is extracted with dichloromethane to obtain crude (4R)-4-propyldihydrofuran-2(3H)-one, which is purified by vacuum distillation to get pure Intermediate 3 i.e. (4R)-4-propyldihydrofuran-2(3H)-one (405 g, 79.63% yield, 91.68% ee). Analytical characterization is carried on formed Intermediate 3B to further confirm the formation of the same with desired attributes.

### Results

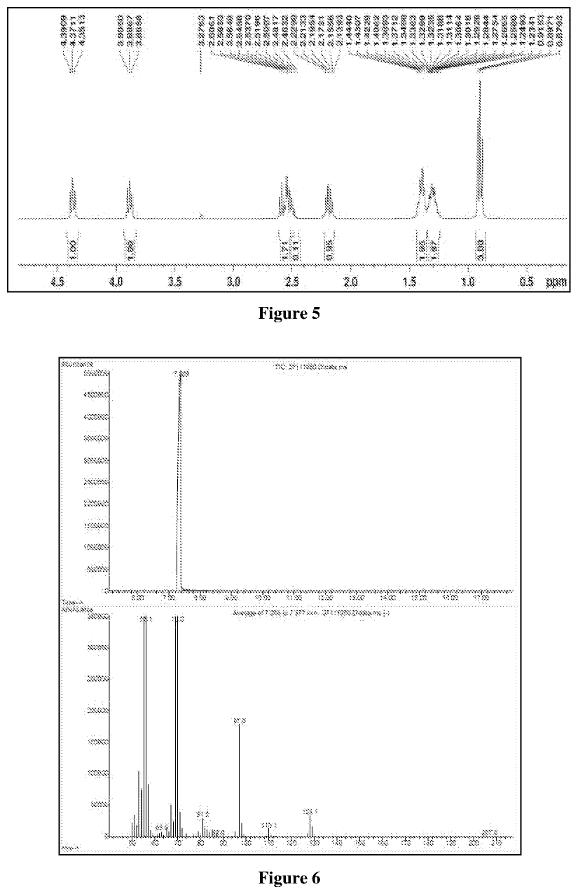

i) The proton NMR data as illustrated in accompanying FIG. 5 depicts the following data: ¹H NMR (400 MHz, DMSO-d₆): δ 0.87 (t, J=7.24 Hz, 3H), 1.27-1.44 (m, 4H), 2.18 (dd, J=16.24 & 7.44 Hz, 1H), 2.44-2.63 (m, 2H), 3.87 (t, J=7.16 Hz, 1H), 4.35 (t, J=7.44 Hz, 1H), that supports formation of Intermediate 3 of the present invention.

ii) The GC-MS analysis data as illustrated in accompanying FIG. 6 depicts the following data: (m/z)=128.1, confirming the formation of Intermediate 3 of the present invention.

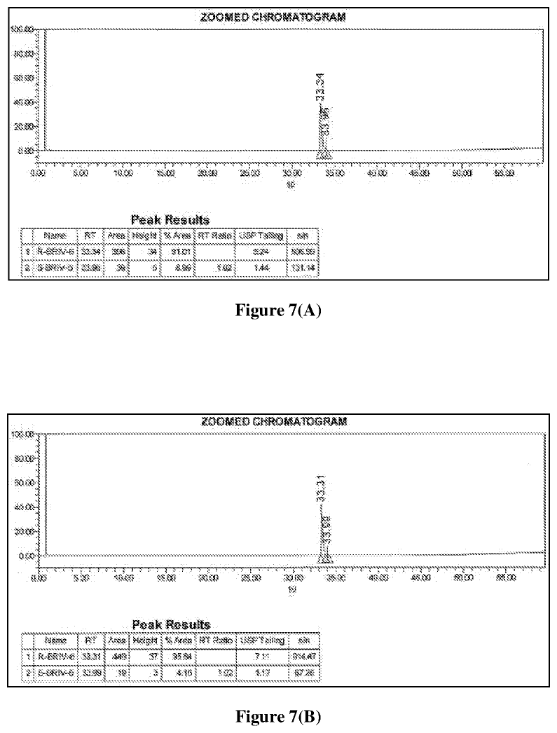

iii) The Chiral GC analysis data of example 3(A) as illustrated in accompanying FIG. 7(A) depicts the following data: R-isomer=91.01% and S-isomer=8.99% and the Chiral GC analysis data of example 3(B) as illustrated in accompanying FIG. 7(B) depicts the following data: R-isomer=95.14%, S-isomer=4.16%.

### Example 4: Synthesis of (3R)/(3S)-3-(hydroxymethyl)-N-[(1S)-1-phenylethyl] hexanamide [Compound/Intermediate-4] (Step-4)

Example 4 illustrates a process for preparing (3R)/(3S)-3-(hydroxymethyl)-N-[(1S)-1-phenylethyl]hexanamide (Compound/Intermediate 4) from Intermediate 3 of example 3 above by the process as developed in the present invention.

### Procedure

In step 4 of the present invention, a mixture of (±)-4-propyldihydrofuran-2-one (Intermediate 3) (100 g, 0.78 mol, 1 eq), triethylamine (163.1 mL, 1.17 mol, 1.5 eq), S-phenylethylamine (211 ml, 1.63 mol, 2.1 eq) and water (in catalytic amount) are refluxed at a temperature between 95-100° C. for 12-18 hrs. The mixture is cooled to room temperature followed by extraction with dichoromethane, which is subsequently washed with aq. HCl; and further concentrated under reduced pressure to obtain Compound/Intermediate 4 i.e. 3-(hydroxymethyl)-N-[(1S)-1-phenylethyl] hexanamide (175 gm, 90% yield). Analytical characterization is carried on formed Intermediate 4 to further confirm the formation of the same with desired attributes.

### Results

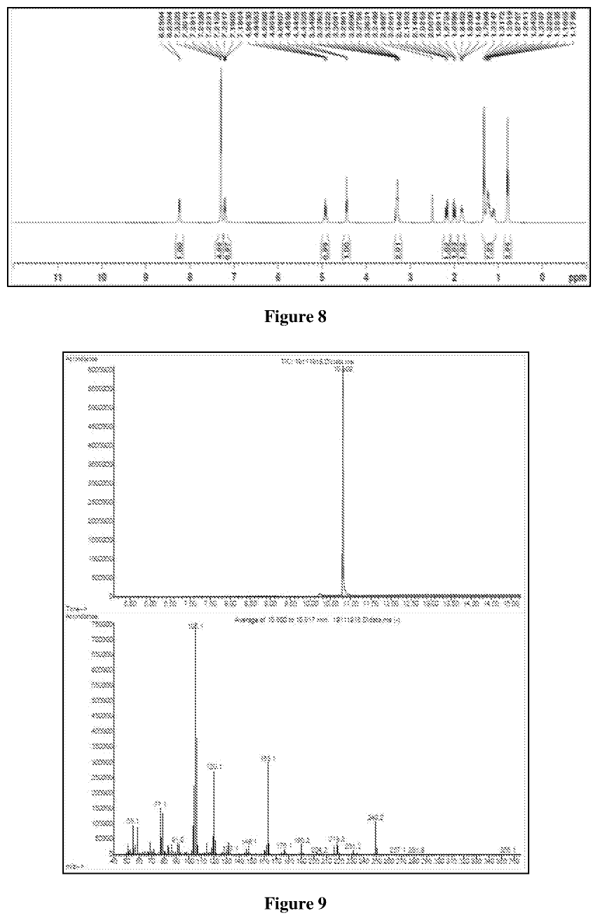

i) The proton NMR analysis as illustrated in accompanying FIG. 8 depicts following data: ¹H NMR (400 MHz, DMSOd₆): δ 0.78 (t, J=7.04 Hz, 3H), 1.04-1.29 (m, 4H), 1.32 (d, J=7.04 Hz, 3H), 1.78-1.85 (m, 1H), 1.92-2.05 (m, 1H), 2.14-2.22 (m, 1H), 3.20-3.32 (m, 2H), 4.43 (t, J=5.16 Hz, 1H), 4.92 (t, J=7.24 Hz, 1H), 7.20-7.22 (d, 1H), 7.29 (d, J=3.88 Hz, 4H), 8.23 (d, J=7.92 Hz, 1H), that supports formation of Intermediate 4 of the present invention.

ii) The GC-MS analysis data as illustrated in accompanying FIG. 9 depicts the following data: (m/z)=249.2, confirming the formation of Intermediate 4 of the present invention.

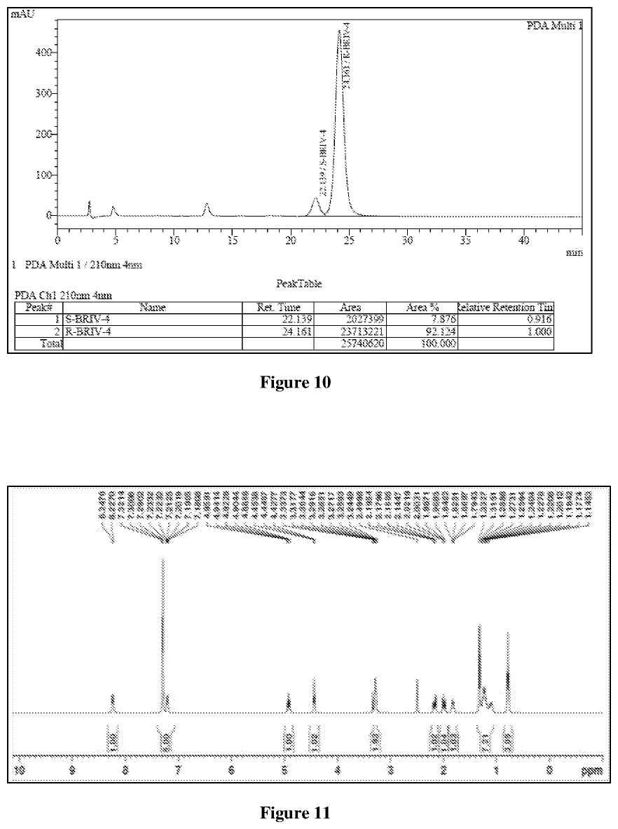

iii) The Chiral HPLC analysis as illustrated in accompanying FIG. 10 depicts the following data: RS isomer=92.124; SS isomer=7.876, which shows the diastereomeric excess of (3R)-3-(hydroxymethyl)-N-[(1S)-1-phenylethyl]hexanamide.

### Example 5: Synthesis of (3R)-3-(Hydroxymethyl)-N-[(1S)-1-Phenylethyl] hexanamide [Compound/Intermediate 5] (Step-5)

Example 5 illustrates the process for preparing (3R)-3-(Hydroxymethyl)-N-[(1S)-1-Phenylethyl]hexanamide (Compound/Intermediate 5) from Intermediate 4 of example 4 above by the process as developed in the present invention.

### Procedure

In step 5 of the present invention, Intermediate 4 (175 g, ˜84% de; 0.7 mol; 1 eq) is added to a mixed solvent of isopropyl acetate and di-isopropyl ether (5:95 volumetric ratio; total 10 vol). The reaction mixture is heated to a temperature of 55-60° C. to get a clear solution, which is further cooled to 0° C. Furthermore, the obtained solid is filtered to get a solid residue, which is subsequently dried to get (3R)-3-(hydroxymethyl)-N-[(1S)-1-phenylethyl]hexanamide i.e. Intermediate 5 (135.0 g, 77% yield, 100% de). Analytical characterization is carried on formed Intermediate 5 to further confirm the formation of the same with desired attributes.

### Results

i) The proton NMR analysis as illustrated in accompanying FIG. 11 depicts the following data: ¹H NMR (400 MHz, DMSO-d₆): δ 0.78 (t, J=6.56 Hz, 3H), 1.05-1.15 (m, 1H), 1.16-1.28 (m, 3H), 1.32 (d, J=6.88 Hz, 3H), 1.76-1.88 (m, 1H), 1.92-2.05 (m, 1H), 2.12-2.20 (m, 1H), 3.28 (br s, 2H), 4.44 (t, J=4.88 Hz, 1H), 4.91 (t, J=7.16 Hz, 1H), 7.13-7.23 (m, 1H), 7.24-7.38 (m, 4H), 8.24 (d, J=7.76 Hz, 1H), that confirms the formation of Intermediate 5 of the present invention.

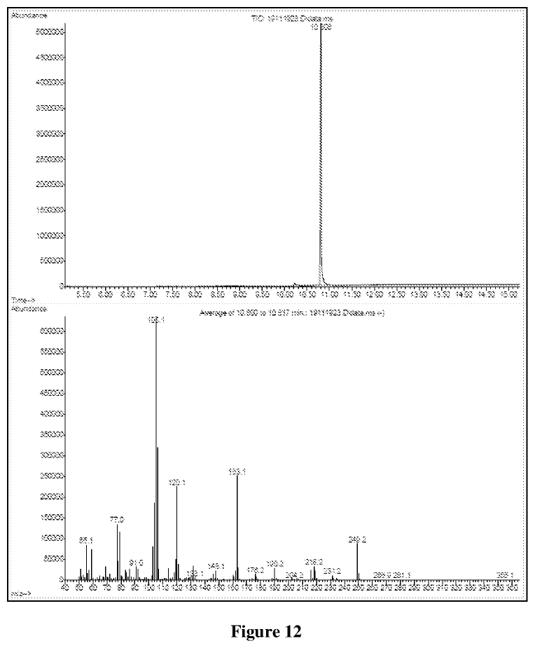

ii) The GC-MS analysis data as illustrated in accompanying FIG. 12 depicts the following data: (m/z)=249.2, confirming the formation of Intermediate 5 of the present invention.

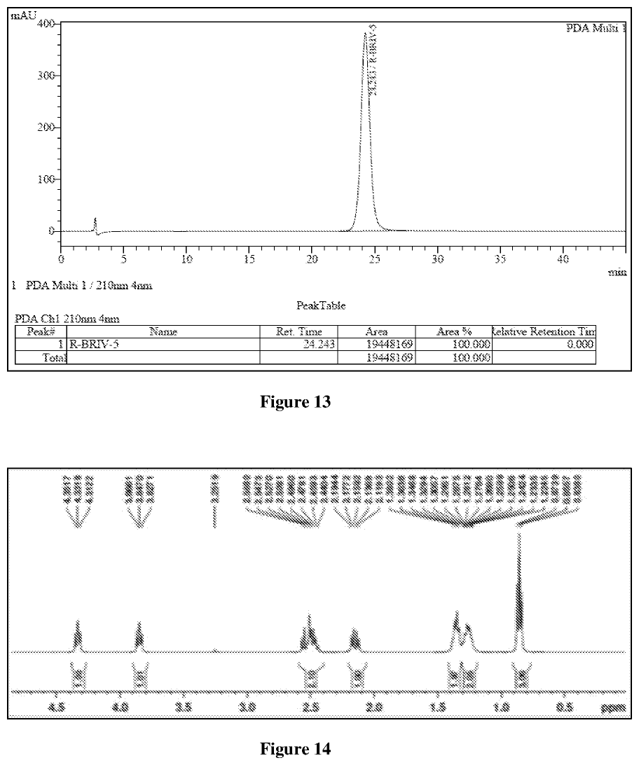

iii) The chiral HPLC analysis as illustrated in accompanying FIG. 13 depicts formation of (3R)-3-(Hydroxymethyl)-N-[(1S)-1-Phenylethyl]hexanamide (Compound/Intermediate 5) with 100% diastereomeric excess.

### Example 6: Synthesis of (R)-4-propyldihydrofuran-2(3H)-one [Compound/Intermediate 6] (Step-6)

Example 6 illustrates the process for preparing (R)-4-propyldihydrofuran-2(3H)-one (Compound/Intermediate 6) from Intermediate 5 of example 5 above by the process as developed in the present invention.

### Procedure

In step 6 of the present invention, Intermediate 5 (600 g, 2.41 mol, 1 eq.) is taken with 30% aq H₂SO₄ (10 vol, 6 L) in a reactor and stirred at 100° C. for 6 hours. The reaction mass is cooled to a temperature between 20-30° C. and then extracted with dichloromethane (3 L, 5 vol). Further, the organic layer is washed with water followed by brine solution (1.2 L, 2 vol), which is subsequently dried over anhydrous Na₂SO₄. The organic layer is concentrated and purified by vacuum distillation to get pure Intermediate 6 i.e. (R)-4-propyldihydrofuran-2(3H)-one as a colourless liquid (295 g, 95% yield, 100% ee). Analytical characterization is carried on formed Intermediate 6 to further confirm the formation of the same with desired attributes.

### Results

i) The proton NMR analysis as illustrated in accompanying FIG. 14 depicts the following data: ¹H NMR (400 MHz, DMSO-d₆): δ 0.87 (t, J=7.24 Hz, 3H), 1.21-133 (m, 2H), 1.35-1.42 (m, 2H), 2.18 (dd, J=8.44 & 24.16 Hz, 1H), 2.45-2.62 (m, 2H), 3.87(dd, J=7.28 & 8.46 Hz, 1H), 4.35 (t, J=8.24 Hz, 1H), which confirms the formation of Intermediate 6 of the present invention.

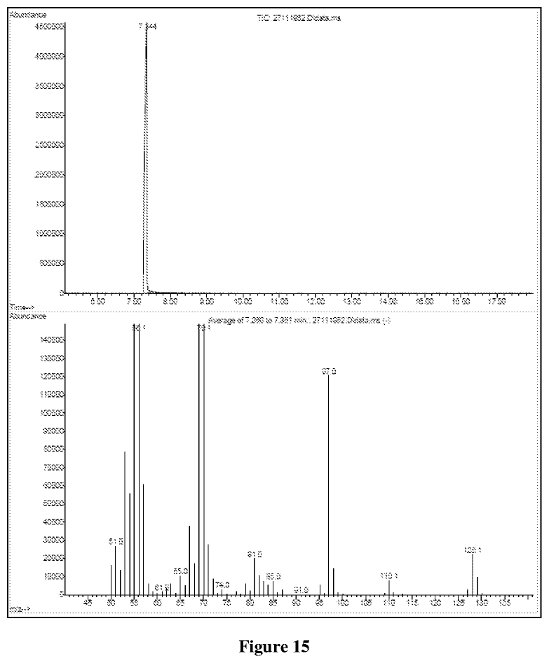

ii) The GC-MS analysis data as illustrated in accompanying FIG. 15 depicts the following data: (m/z)=128.1, confirming the formation of Intermediate 6 of the present invention.

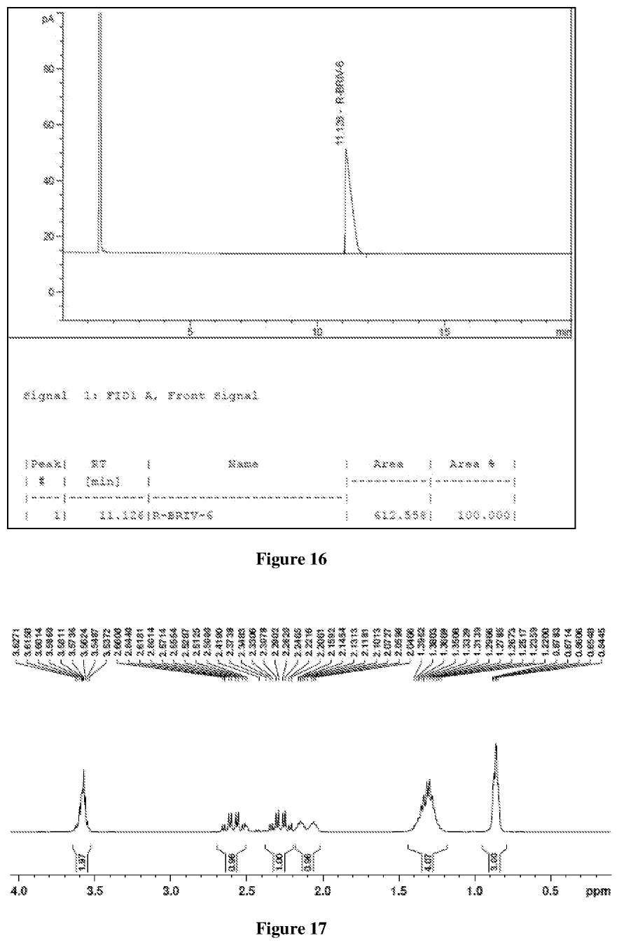

iii) The chiral GC analysis as illustrated in accompanying FIG. 16 depicts the following data: Enantiomeric ratio (R:S): 100:0, confirming the formation of (R)-4-propyldihydrofuran-2-one i.e. Intermediate 6 of the present invention.

### Example 7: Synthesis of (3R)-3-(bromomethyl)hexanoic acid [Compound/Intermediate 7] (Step-7)

Example 7 illustrates the process for preparing (3R)-3-(bromomethyl)hexanoic acid (Compound/Intermediate 7) from Intermediate 6 of example 6 above by the process as developed in the present invention.

### Procedure

In step 7 of the present invention, hydrogen bromide (33% w/w solution) in acetic acid (678.12 mL, 3744.88 mmol, 4 eq. is charged to a 1 L round bottom flask equipped with a condenser and a sodium hydroxide scrubber) under a nitrogen flow. The solution is cooled to 5° C. to which a solution of Intermediate 6 (120 g, 936.32 mmol, 1 eq) in acetic acid (60 mL) is slowly added, while maintaining the temperature below 5° C. over a period of 20 min. The solution is warmed to room temperature and further heated at 80° C. for 2.5 h. Furthermore, the reaction mixture is cooled to 20° C., diluted with water (600 mL) and extracted twice with dicholoromethane (600 mL). The combined organic phases is washed thrice with water (360 mL), which is further dried over anhydrous sodium sulfate and concentrated to get Intermediate 7 i.e. (3R)-3-(bromomethyl)hexanoic acid as a pale yellow oil (180 g, 91.95%). Analytical characterization is carried on formed Intermediate 7 to further confirm the formation of the same with desired attributes.

### Results

i) The proton NMR analysis as illustrated in accompanying FIG. 17 depicts the following data: ¹H NMR (400 MHz, CDCl₃): δ 0.92 (t, J=7.04 Hz, 3H), 1.22-1.54 (m, 4H), 2.12-2.25 (m, 1H), 2.39 (dd, J=5.92 & 16.42 Hz, 1H), 2.56 (dd, J=7.28 & 16.48 Hz, 1H), 3.48 (dd, J=5.12 & 10.26 Hz, 1H), 3.57 (m, J=4.04 & 10.28 Hz, 1H), 12.71 (s, 1H), which confirms the formation of Intermediate 7 of the present invention.

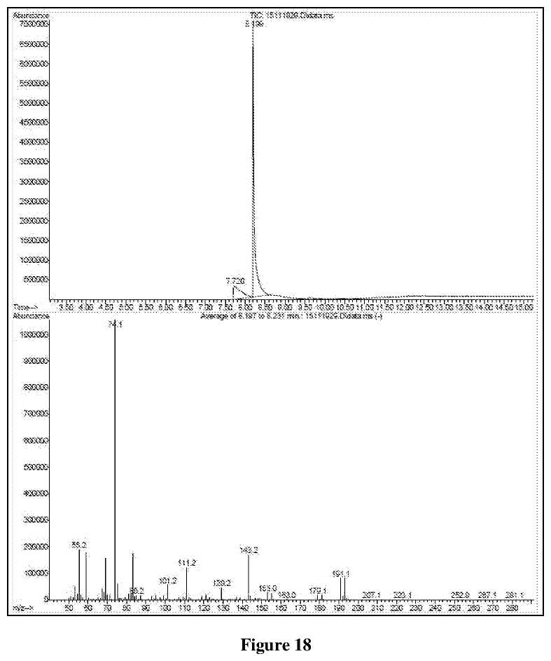

ii) GC-MS (m/z) analysis as illustrated in accompanying FIG. 18 depicts the following data: m/z=207.1, confirms the formation of Intermediate 7 of the present invention.

### Example 8: Synthesis of ethyl (3R)-3-(bromomethyl)hexanoate [Compound/Intermediate 8] (Step-8)

Example 8 illustrates the process for preparing (3R)-3-(bromomethyl)hexanoate (Compound/Intermediate 8) from Intermediate 7 of example 7 above by the process as developed in the present invention.

### Procedure

In step 8 of the present invention, said Intermediate 7 i.e. (3R)-3-(bromomethyl) hexanoic acid (100 g, 95.65 mmol, 1 eq) and ethanol (400 mL) is taken in a condenser equipped round bottom flask, where the reaction mixture is kept under a nitrogen atmosphere at 25° C. Further, Hydrochloric acid (37% w/w) (10 mL, 119.55 mmol, 0.25 eq) is added to the reaction mixture with subsequent heating at 40° C. for 24 h. Furthermore, ethanol is removed by evaporation and the reaction mass is extracted in ethyl acetate (400 mL). The organic phase is successively washed with aqueous sodium hydroxide solution (10 g, 239.145 mmol in 150 mL of water). Then the organic phase is dried over anhydrous Na₂SO₄ and subsequently concentrated to obtain Intermediate 8 of the present invention i.e. ethyl (3R)-3-(bromomethyl)hexanoate (95 g, 83.76%) as a pale yellow oil. ¹H-NMR and GC-MS studies of said Intermediate 8 are performed.

### Results

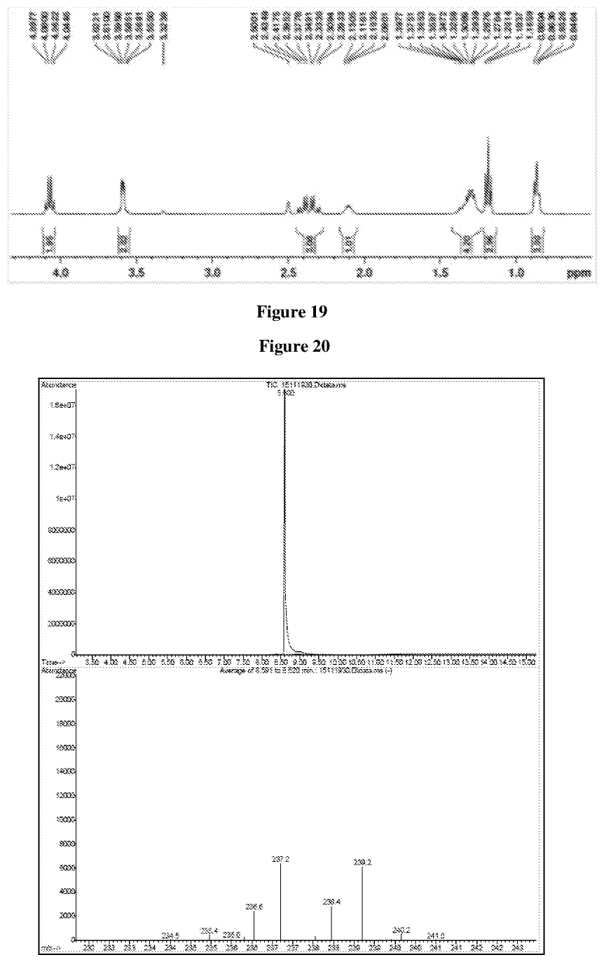

i) The proton NMR analysis as illustrated in accompanying FIG. 19 depicts the following data: ¹H NMR (400 MHz, CDCl₃): δ 0.90 (t, J=7.04 Hz, 3H), 1.20 (t, J=7.20 Hz, 3H), 1.27-1.39 (m, 4H), 2.12-2.13 (m, 1H), 2.32 (dd, J=5.92 & 15.98 Hz, 1H), 2.46 (dd, J=7.32 & 16.0 Hz, 1H), 3.58 (dd, 2H), 4.08 (q, J=7.04 Hz, 2H), which confirms the formation of Intermediate 8 of the present invention.

ii) GC-MS analysis as illustrated in accompanying FIG. 20 depicts the following data: m/z=237.2 and 239.2, which confirms the formation of Intermediate 8 of the present invention.

### Example 9: Synthesis of (2S)-2-[(4R)-2-oxo-4-propylpyrrolidin-1-yl]butanamide or Brivaracetam

Example 9 illustrates the process for preparing (2S)-2-[(4R)-2-oxo-4-propylpyrrolidin-1-yl]butanamide (Brivaracetam) from Intermediate 8 of example 8 above by the process as developed in the present invention.

### Procedure

In steps 9-10 of the present invention, Intermediate 8 (4.5 g, 18.97 mmol, 1 eq), (2S)-2-aminobutanamide (2.91 g, 28.49 mmol, 1.5 eq), tetrabutylammonium iodide (2.103 g, 5.69 mmol, 0.3 eq), sodium carbonate (4.023 g, 37.952 mmol, 2.0 eq) and isopropyl acetate (22 mL, 4.88 vol) are taken in a round bottom flask. The suspension is refluxed for 28 hrs and subsequently cooled to 15° C. The suspension is filtered at 15° C. and washed with isopropyl acetate (4.5 mL, 1 vol). The filtrate is then charged into another round bottom flask and further diluted with isopropyl acetate (18 mL, 4 vol), which is then heated to 60° C. Furthermore, Acetic acid (0.816 mL, 14.232 mmol, 0.75 eq) is added slowly over a period of 20 min and then the reaction mixture is agitated at 60° C. for 1 h 30 min. The suspension is cooled to 25° C. and filtered. The reaction mass is washed with isopropyl acetate (4.5 mL, 1 vol). Water (4.5 mL, 1 vol) is added to it followed by addition of sodium bicarbonate (800 mg) till to attend pH 7 for the aqueous layer, after which the aqueous and organic phases are separated. The aqueous layer is further washed with water (4.5 mL, 1 vol). The organic phase is evaporated to give crude product (4.3 g) as a yellow gummy solid. A blended crude product (4.3 g) is partially dissolved in methyl tert-butylether (43 mL) into a 100 mL RBF and agitated at 25° C. for 20 h under a nitrogen atmosphere. The insoluble solid is eliminated by filtration and the filtrate is evaporated to a crude (3.9 g) as a yellow gummy solid. After complete dissolution in isopropyl acetate (2.15 mL), the mass temperature is set at 36° C. and the suspension is cooled down to 23° C. in 20 minutes, further to 15° C. in 1 hour on stirring for 15 min at 15° C. The product is isolated by filtration and the obtain mass is washed with isopropyl acetate (1.5 mL and 0.65 mL). The product is then dried at 40° C. under vacuum to afford the desired Brivaracetam (510 mg, 12.66%) as white solid. This final compound is analysed by ¹H-NMR, LC-MS, GC-MS and chiral HPLC.

### Results

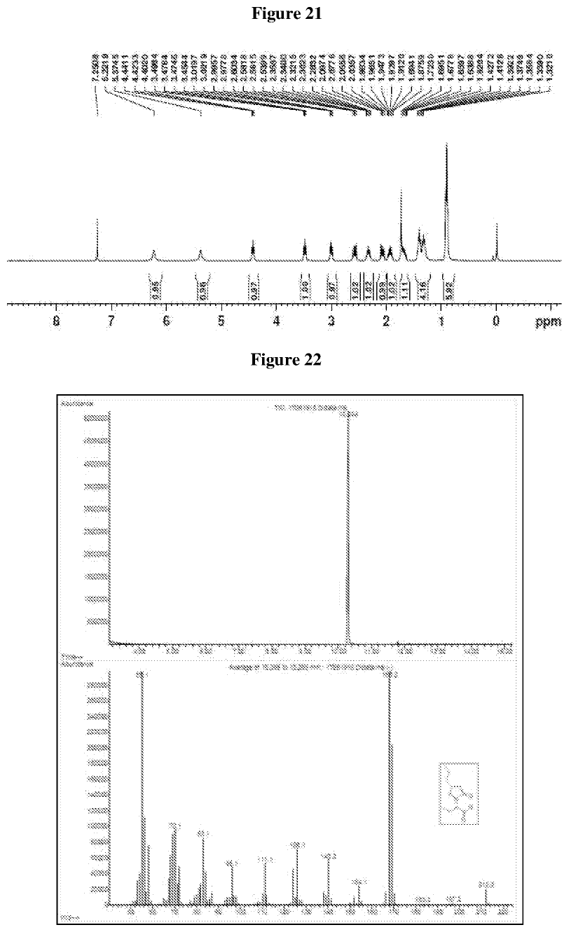

i) The proton NMR analysis as illustrated in accompanying FIG. 21 depicts the following data: ¹H NMR (400 MHz, CDCl₃): δ 0.76-0.98 (m, 6H), 1.19-1.48 (m, 4H), 1.61-1.72 (m, 1H), 1.84-1.98 (m, 1H), 2.06 (dd, J=7.92 & 16.74 Hz, 1H), 2.23-2.42 (m, 1H), 2.57 (dd, J=8.64 & 16.76 Hz, 1H), 2.99 (dd, J=7.12 & 9.62 Hz, 1H), 3.47 (dd, J=8.0 & 9.56 Hz, 1H), 4.42 (t, J=7.12 Hz, 1H), 5.37 (br s, 1H), 6.22 (br s, 1H), which confirms the formation of Brivaracetam from Intermediate 8.

ii) The GC-MS analysis as illustrated in accompanying FIG. 22 depicts the following data: (m/z)=212.2, which further confirm the formation of Brivaracetam.

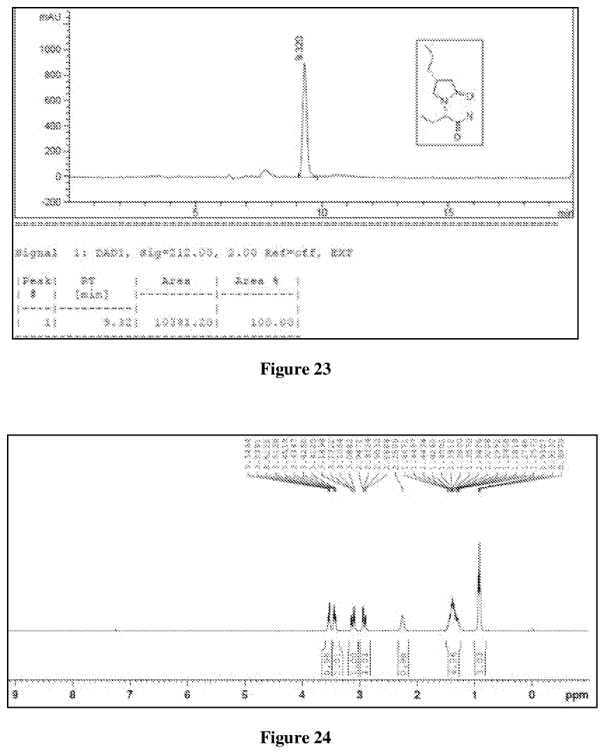

iii) The chiral HPLC analysis as illustrated in accompanying FIG. 23 depicts the following data: 100% diastereomeric excess of Brivaracetam

### Example 10: Synthesis of (3R)-3-(bromomethyl)pentanoyl chloride (Compound/Intermediate 9)

Example 10 illustrates the process for preparing (3R)-3-(bromomethyl)pentanoyl chloride (Compound/Intermediate 9) from Intermediate 7 of example 7 above by the process as developed in the present invention.

### Procedure

In step 11 of the present invention, Intermediate 7 (74 g, 0.354 mol, 1 eq) is charged into a round bottom flask and cooled to 0° C. temp followed by addition of Thionyl chloride (SOCl₂) (52.65 mL, 0.708 mmol). Further, the reaction mass is heated to reflux at 70° C. for 1 h. Furthermore, SOCl₂ is distilled out to obtain crude (3R)-3-(bromomethyl)pentanoyl chloride, which is subsequently purified by vacuum distillation to get pure Intermediate 9 (73 g, 90.62% yield). Analytical characterization is carried on formed Intermediate 9 to further confirm the formation of the same with desired attributes.

### Results

The proton NMR analysis as illustrated in accompanying FIG. 24 depicts the following data: ¹H-NMR (400 MHz, CDCl₃) of the title compound: δ3.41-3.54 (2H, m), δ 3.08-3.14 (1H, m), δ2.88-2.94 (1H, m), δ2.25 (1H, m), δ1.23-1.46 (4H, m), δ0.91 (3H, t), which confirms the formation of (3R)-3-(bromomethyl)pentanoyl chloride i.e. Intermediate 9.

### Example 11: Synthesis of (2S)-2-[(4R)-2-oxo-4-propylpyrrolidin-1-yl] butanamide or Brivaracetam

Example 11 illustrates the process for preparing (2S)-2-[(4R)-2-oxo-4-propylpyrrolidin-1-yl]butanamide or Brivaracetam from Intermediate 9 of example 10 above by the process as developed in the present invention.

### Procedure

In steps 12-13 of the present invention, (S)-2-aminobutanamide hydrochloride (1.34 g, 9.69 mmol) is added to dichloromethane (40 ml) and subsequent addition of N,N-Diisopropylethylamine (3.223, 18.5 mmol) to the solution at room temperature, which is followed by stirring for 30 min. Intermediate 9 (2.0 g, 8.81 mmol) is then added to the reaction mixture. After completion of the reaction, water (30 ml) and ethanol (4 ml) are added to the reaction mixture. The mixture is extracted twice with dichloromethane (80 mL). The combined organic layer is washed with brine and dried over anhydrous Na₂SO₄. Further, the reaction mass is filtered and solvent is evaporated to obtain crude (3R)-3-(bromomethyl)-N-[(1S)-1-carbamoylpropyl] hexanamide, which is furthermore purified by trituration to get (3R)-3-(bromomethyl)-N-[(1S)-1-carbamoylpropyl] hexanamide (1.3 g, 50.32% yield).

The crude compound of (3R)-3-(bromomethyl)-N-[(1S)-1-carbamoylpropyl] hexanamide (1.0 g, 3.4 mmol) is added to anhydrous tetrahydrofuran (15 ml). Then, t-BuOK (0.459 g, 4.09 mmol) is added to the reaction mass at −30° C. After completion of the reaction, the saturated NH₄CI solution (10 ml) is added to the solution, which is further extracted thrice with ethyl acetate (10 ml). The combined organic layers are then washed with brine and dried over anhydrous Na₂SO₄. The reaction mass is filtered and solvent is evaporated to get Brivaracetam (69.06% yield, 500 mg). Analytical characterization is carried on formed final compound Brivaracetam to further confirm the formation of the same with desired attributes.

### Results

The proton NMR analysis as illustrated in accompanying FIG. 25 depicts the following data: ¹H NMR (400 MHz, CDCl₃): δ 0.76-0.98 (m, 6H), 1.19-1.48 (m, 4H), 1.61-1.72 (m, 1H), 1.84-1.98 (m, 1H), 2.06 (dd, J=7.92 & 16.74 Hz, 1H), 2.23-2.42 (m, 1H), 2.57 (dd, J=8.64 & 16.76 Hz, 1H), 2.99 (dd, J=7.12 & 9.62 Hz, 1H), 3.47 (dd, J=8.0 & 9.56 Hz, 1H), 4.42 (t, J=7.12 Hz, 1H), 5.37 (br s, 1H), 6.22 (br s, 1H), which confirms the formation of Brivaracetam from Intermediate 9.

### Example 12: Synthesis of (R)-3-(chloromethyl)hexanoyl chloride (Compound/Intermediate 10)

Example 12 illustrates the process for preparing (R)-3-(chloromethyl)hexanoyl chloride (Compound/Intermediate 10) from Intermediate 6 of example 6 above by the process as developed in the present invention.

### Procedure

In step 14 of the present invention, a reaction mixture of thionyl chloride (40 ml), anhydrous zinc chloride (ZnCl₂) (4 g, 31.2 mmol), and Intermediate 6 (20 g, 156 mmol) are stirred at 85° C. The solvent is then evaporated in vacuum and the residue is subsequently purified by vacuum distillation to obtain the compound 10 as a yellow oil (19 g, 66.5% yield). Analytical characterization is carried on formed Intermediate 10 to further confirm the formation of the same with desired attributes.

### Results

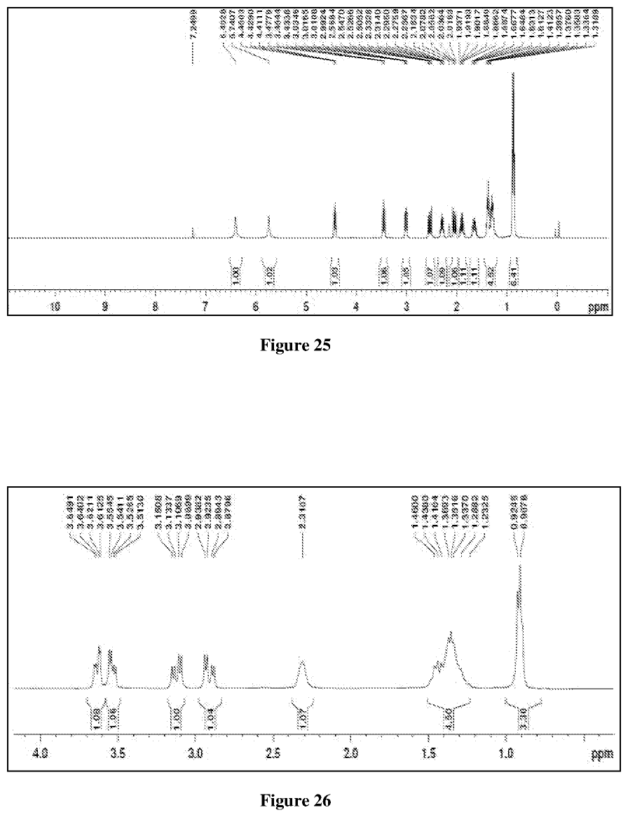

The proton NMR analysis as illustrated in accompanying FIG. 26 depicts the following data: ¹H-NMR (400 MHz, CDCl₃): δ3.63 (1H, dd), δ3.53 (1H, dd), δ3.12 (1H, dd), δ2.91 (1H, dd), δ2.31 (1H, m), δ1.23-1.46 (4H, m), δ0.92 (3H, t), which confirms the formation of Intermediate 10 of the present invention.

### Example 13: Synthesis of (2S)-2-[(4R)-2-oxo-4-propylpyrrolidin-1-yl]butanamide or Brivaracetam

Example 13 illustrates the process for preparing (2S)-2-[(4R)-2-oxo-4-propylpyrrolidin-1-yl]butanamide or Brivaracetam from Intermediate 10 of example 12 above by the process as developed in the present invention.

### Procedure

In steps 15-16 of the present invention, (S)-2-aminobutanamide hydrochloride (3.34 g, 24 mmol) is added to dichloromethane (60 ml) followed by addition of triethylamine (4.86 g, 48 mmol) at room temperature with constant stirring for 30 min. Compound 10 (4.0 g, 21.6 mmol) is then added to the solution. After completion of the reaction, water (30 ml) and ethanol (4 ml) are added to the reaction mixture. Further, the mixture is extracted twice with dichloromethane (80 mL). The combined organic layers are washed with brine and subsequently dried over anhydrous sodium sulphate (Na₂SO₄), which is followed by evaporation of the solvent to obtain crude product of (R)-N-((S)-1-amino-1-oxobutan-2-y1)-3-(chloromethyl)hexanamide (5.25 g, 96.7% yield).

Furthermore, the crude compound (R)-N-((S)-1-amino-1-oxobutan-2-yl)-3-(chloromethyl)hexanamide (5.0 g, 20 mmol) is added to anhydrous tetrahydrofuran (75 ml) followed by addition of t-BuOK (2.8 g, 25 mmol) to the said reaction mixture. After completion of the reaction saturated NH₄CI solution (25 ml) is added to the reaction mass. Further, the reaction mass is extracted thrice with ethyl acetate (25 ml). Furthermore, the combined organic layers are washed with brine and subsequently dried over anhydrous Na₂SO₄, which is followed by filtration and evaporation of the solvent to obtain Brivaracetam (3.96 g, 93% yield). The compound is analysed by ¹H-NMR. Analytical characterization is carried on formed Brivaracetam to further confirm the formation of the same with desired attributes.

### Results

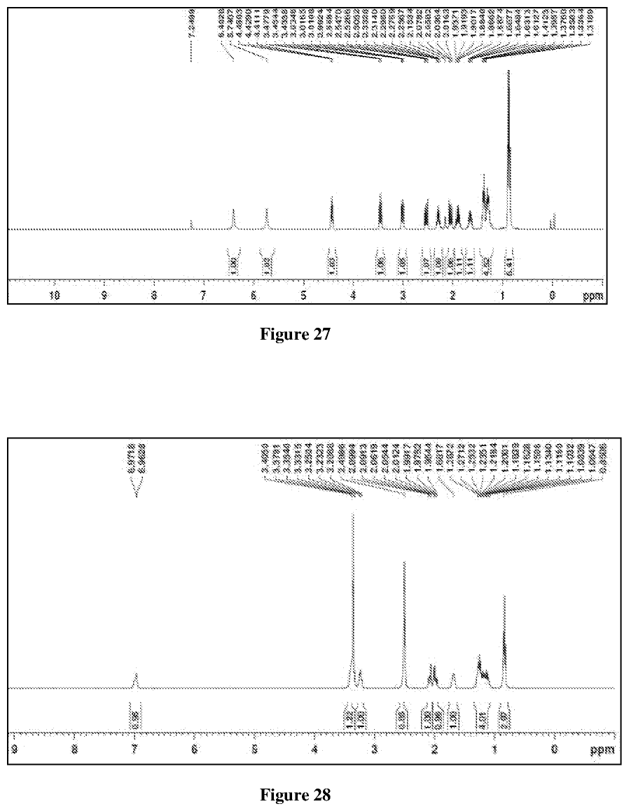

The proton NMR analysis as illustrated in accompanying FIG. 27 depicts the following data: ¹H NMR (400 MHz, CDCl₃): δ 0.76-0.98 (m, 6H), 1.19-1.48 (m, 4H), 1.61-1.72 (m, 1H), 1.84-1.98 (m, 1H), 2.06 (dd, J=7.92 & 16.74 Hz, 1H), 2.23-2.42 (m, 1H), 2.57 (dd, J=8.64 & 16.76 Hz, 1H), 2.99 (dd, J=7.12 & 9.62 Hz, 1H), 3.47 (dd, J=8.0 & 9.56 Hz, 1H), 4.42 (t, J=7.12 Hz, 1H), 5.37 (br s, 1H), 6.22 (br s, 1H), which confirms the formation of Brivaracetam from Intermediate 10.

### Example 14: Synthesis of 3-(hydroxymethyl)hexanoyloxy sodium (Compound/Intermediate 11)

Example 14 illustrates the process for preparing 3-(hydroxymethyl) hexanoyloxy sodium (Compound/Intermediate 11) from Intermediate 6 of example 6 above by the process as developed in the present invention.

### Procedure

In step 17A of the present invention, Intermediate 6 (10 g, 0.078 mole) is taken in aq. sodium hydroxide (6.25 gm, 0.16 mole) solution and stirred for 2 hours. The reaction mass is then filtered to get 3-(hydroxymethyl) hexanoyloxy sodium (12.8 gm, 95% yield) i.e. Intermediate 11 of the present invention. Analytical characterization is carried on formed Intermediate 11 to further confirm the formation of the same with desired attributes.

### Results

The proton NMR analysis as illustrated in accompanying FIG. 28(A) depicts the following data: ¹H NMR (400 MHz, CDCl₃): δ0.85 (t, 3H), 1.10-1.29 (m, 4H), 1.68 (br, s, 1H), 1.95-2.10 (m, 2H), 2.49 (s, 1H), 3.20-3.26 (t, 1H), 3.33-3.41 (m, 1H), 6.96-6.98 (t, 1H), which confirms the formation of Intermediate 11 from Intermediate 6.

### Example 15: Synthesis of 3-(hydroxymethyl) hexanoyloxy lithium (Compound/Intermediate 11)

Example 15 illustrates the process for preparing 3-(hydroxymethyl)hexanoyloxy lithium (Compound/Intermediate 11) from Intermediate 5 as shown in above example 5 by the process as developed in the present invention.

### Procedure

In step 17B of the present invention, Intermediate 5 (10 g, 0.04mole, 1 eq.) is taken in 30% Water/THF (100 ml, 10 v) solution mix in a sealed tube. LiOH (1.92 gm, 0.08 mole, 2 eq.) and 18-Crown-6 (1.06 g, 0.004 mole, 0.1 eq.) are then added to the said reaction mass and heated to 95-100° C. and maintained for 24 hours. The reaction mass is concentrated to remove THF and the aq. layer is washed with heptane. Finally, the aqueous layer is evaporated to get Intermediate 11. Analytical characterization is carried on formed Intermediate 11 to further confirm the formation of the same with desired attributes.

### Results

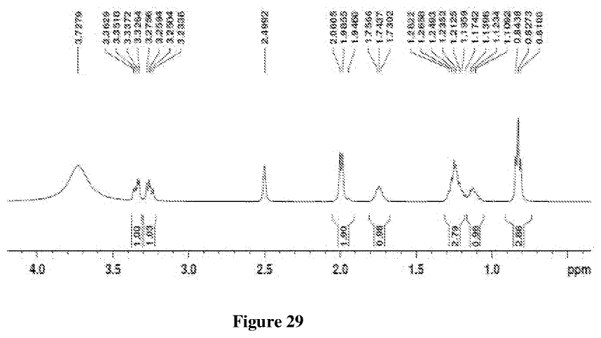

The proton NMR analysis as illustrated in accompanying FIG. 29 depicts the following data: ¹H NMR (400 MHz, DMSO): δ0.80-0.85 (t, 3H), 1.10-1.29 (m, 4H), 1.73-1.76 (m, 1H), 1.95-2.10 (m, 2H), 2.49 (s, 1H), 3.20-3.26 (t, 1H), 3.33-3.41 (m, 1H), 3.72 (br, s, 1H), which confirms the formation of Intermediate 11 from Intermediate 5.

### Example 16: A Comparative Data of the Present Invention in View of the Closest Prior Arts

Example 16 provides a comparative data of the present invention in view of the closest reported prior arts.

The following data shows that the present invention is not only economical, industrially scalable, but also provides Brivaracetam and its key intermediates with superior yields and chiral purity.

Thus the salient features and the results obtained by the present invention have been compared with those reported in the closest prior arts (I, II and/or III) as depicted in the following table 1:—

I. Gregory Hughes et al, J. Am. Chem. Soc., 2003, v. 125, p. 11253-11258 reports acceleration in the rate of an enantioselective copper-catalyzed conjugate reduction of α, β-unsaturated lactones, upon addition of alcohol additives (e.g. EtOH, PrOH). Good to excellent yields and enantioselectivities are realized while reduction of 3-R-substituted butenolide (R=Ph, Bn,n-Bu, i-Pr) in presence of said additive by means of using a catalyst generated in situ from CuCl (5 mol %), t-BuONa (5-20 mol %), (S)-p-tol-BINAP (5 mol %) and a silane (PMHS or Ph₂SiH₂) (4 equiv) at −15 to −40° C. temperature with isolated yield upto 34-50% and around 80-91 ee %.

II. WO2018042393 discloses the enantioselective preparation of Brivaracetam using chiral auxiliary, S-4-phenyloxazolidine-2-one and valeryl chloride, followed by alkylation with tert-butylbromoacetate using LIHMDS as base at lower temperature (−55 to −60° C.) to produce an enantiomerically pure isomer which when hydrolyzed followed by reduction with a produced acid and BH3.DMS, cyclization of the ester-alcohol intermediate takes place with TFA forming the key Brivaracetam intermediate i.e. (R)-dihydro-4-propylfuran-2(3H)-one.

III. WO2016191435A1 relates to a process fora scalable synthesis of enantiomerically pure Brivaracetam, and related derivatives. It discloses a process for synthesis of the key intermediate of Brivaracetam, (4R)-4-Propyldihydrofuran-2(3H)-one, wherein (R)-(−)-Epichlorohydrin and dialkyl malonate ester are used as the starting materials, followed by Grignard reaction (with ethylmagnesium bromide) at low temperature (−30° C.) in presence of copper (I) iodide catalyst.

TABLE 1
Comparative Data

| | Specific | | Chirality incorporation |
|---|---|---|---|
| Closest | features/ | As disclosed | step (3) and/or other |
| prior | results for | in Closest | steps as used in the |
| arts | comparison | Prior arts | Present Invention |
| I | Amount of | 5 mol % | Lower loading of chiral |
| | Chiral ligand | | ligand i.e. only 0.1-1 |
| | used in the | | mol % |
| | enantioselective | | |
| | reaction | | |
| | Reaction | Cryogenic conditions | Advantageously all |
| | temperature | essentially required | reactions are conducted |
| | | rendering it to be | at ambient temperature |
| | | highly expensive and | (10-35° C.) |
| | | industrially unsuitable. | |
| | Isolated | 34 to 50% | desired R-Lactone |
| | Yield | | (intermediate 3) is |
| | | | obtained in 90-95% |
| | | | yield (better yield). |
| | Enantiomeric | 80 to 91% ee | Comparably better |
| | excess (% ee) | | ~99.90-100% ee |
| | Reaction time | upto 48 hours | Shorter time 5 to 10 |
| | | | hours |
| II | Chiral | Use of expensive | Less expensive reagents |
| | reagents | reagent like S-4 | are used such as (S) |
| | used | phenyloxazolidine-2 | BINAP; thus better for |
| | | one and valeryl | industrial scale-ups |
| | | chloride | |
| | Reaction | Cryogenic conditions | Advantageously |
| | parameters | essentially required | reaction is conducted at |
| | | rendering it to be | ambient temperature |
| | | highly expensive and | (10-35° C.) |
| | | industrially | |
| | | unsuitable. | |
| III | Reaction | Alkyl (4R)-2-oxo-4 | The current process |
| | parameters | propyltetrahydrofuran- | does not require any |
| | | 3-carboxylate is used | decarboxylating agent |
| | | in order to | and/or high boiling |
| | | decarboxylate the | polar solvents. |
| | | produced | Further, reaction |
| | | intermediate; which | surprisingly occurs at |
| | | disadvantageously | ambient temperature, |
| | | requires high | in lesser time, |
| | | temperature (~200° C.) | producing the |
| | | and a high boiling | intermediate 5 and/or 6 |
| | | polar solvent (e.g. | i.e. (R)-lactone with |
| | | DMSO, DMF, NMP). | 99.90-100% ee. |

Therefore, from the comparative data in above table 1, it is evident that the currently developed process comprises of improved and economical reaction parameters and at the same time produces surprisingly superior results than those reported in the prior arts, in terms of specifically obtaining the key intermediates 5 and 6 with 99.90-100% chiral purity. This in turn helps in obtaining Brivaracetam with better yield and superior chiral purity.

### The Overall Advantages of the Present Invention are Provided Below

i) The currently developed process is economical since it requires a low chiral ligand loading of only 0.1 to 1.0 mol % in order to achieve 90-95% R-Lactone.

ii) A novel chirally pure diastereomeric compound (Intermediate 5) is synthesized which is of 99.90-100% chiral purity.

ii) The chiral ligand such as S-BINAP as used in the currently developed process is commercially available and cheap with respect to other reported chiral ligands.

iii) The currently developed process for synthesizing Intermediate 3 is advantageously conducted at a lesser reaction time such as 5 to 10 hours.

iv) The current process is advantageously conducted at an ambient temperature range of 10° C. and 35° C., whereas the reported methods in prior arts disclose requirement of cryogenic conditions for the same reaction (e.g. −45° C. to −25° C.).

v) Brivaracetam is produced by the currently developed process with 99-100% chiral purity and a superior yield of 70-80%.

We claim:

1. A process for enantioselective synthesis of the compound of formula I and key intermediates thereof comprising the steps of: 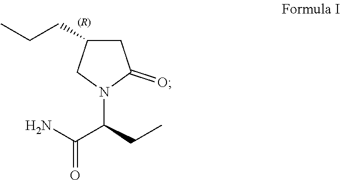
(a) condensing a pentanal with a glycoxylic acid in presence of a condensing agent to form Intermediate 1 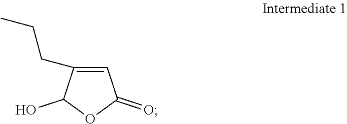
(b) reducing said Intermediate 1 with a reducing agent to form Intermediate 2 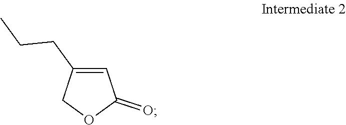
(c) treating said Intermediate 2 with 0.1-1 mol % of a chiral ligand in presence of a metal based catalyst; followed by enantio-selectively reducing the unsaturated lactone of said Intermediate 2 in presence of a reductant and an additive in order to form Intermediate 3 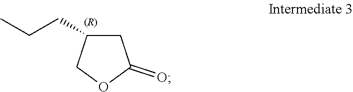
(d) reacting the said Intermediate 3 with a chiral amine in a solvent to produce a diastereomeric mixture of Intermediate 4 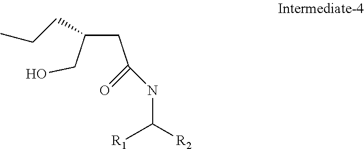
wherein R1 is a substituted or unsubstituted aryl or heteroaryl; and
R2 is a substituted or unsubstituted alkyl or cycloalkyl;
(e) purifying said Intermediate 4 by crystallization in order to form Intermediate 5 having 99.90-100% chiral purity 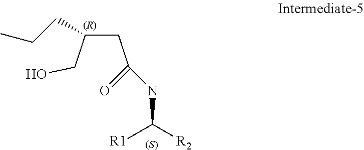
wherein R1 is a substituted or unsubstituted aryl or heteroaryl; and
R2 is a substituted or unsubstituted alkyl or cycloalkyl;
(f) cyclizing said Intermediate 5 with a cyclizing agent in order to form intermediate 6 having an enantiomeric purity of 99.90-100% 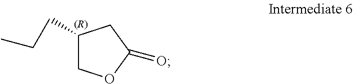
(g) reacting said intermediate 6 with a suitable ring-opening agent to produce the compound intermediate 7A 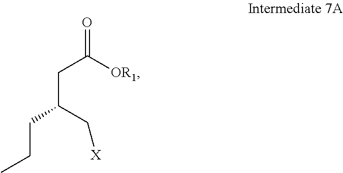
wherein R₁ is selected from saturated or unsaturated C₁₋₂₀ alkyl, substituted or unsubstituted C₁₋₁₀ aryl, a metal of Group I of the Periodic table; and
X is CI, Br, I, OH, OMs, OTs, ONs; with a proviso that X is OH only when R1 is a metal of Group I of the Periodic table;
OR
the compound intermediate 7B: 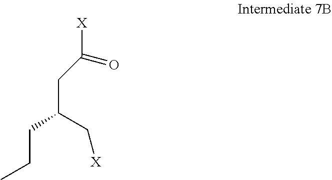
wherein X is selected from a group consisting of CI, Br, I, OMs, OTs, ONs; followed by
(h) reacting the said intermediate 7A OR intermediate 7B with a chiral amide to produce the compound Brivaracetam of formula I with chiral purity of 99-100%.

2. A process for synthesizing Intermediate 3 from Intermediate 2 comprising steps of: 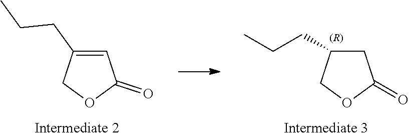
treating Intermediate 2 with a chiral ligand in a loading amount ranging between 0.1 and 1 mol %. in presence of a metal-based catalyst; followed by
enantioselectively reducing the unsaturated lactone of said Intermediate 2 in presence of a reductant and an additive to form Intermediate 3.

3. The process as claimed in claim 1 or claim 2, wherein the metal based catalyst in the step of forming said Intermediate 3 is selected from CuI, CuCl, CuCl₂, Cu(OAc)₂, CuO, Cu(NO₃)₂ or CuBr.

4. The process as claimed in claim 1 or claim 2, wherein the chiral ligand in the step of forming said Intermediate 3 is selected from a group consisting of S-BINAP, S-tol-BINAP, S-BIPHEMP and (R)-SEGPHOS, preferably S-BINAP.

5. The process as claimed in claim 1 or claim 2, wherein the reductant in the step of forming said Intermediate 3 is selected from a group consisting of PMHS (polymethylhydrosiloxane), 1,1,3,3-Tetramethyldisiloxane, Et₃SiH (triethyl siliane) and Ph₂SiH₂ (diphenylsilane).

6. The process as claimed in claim 1 or claim 2, wherein the additive in the step of forming said Intermediate 3 is selected from water, methanol, ethanol, propanol, pentanol, t-butanol, n-butanol, amyl alcohol, isopropyl alcohol and mixtures thereof.

7. The process as claimed in claim 1 or claim 2, wherein reaction in the step of forming said Intermediate 3 is conducted at a temperature ranging between −10° C. and 40° C., preferably between 10° C. and 35° C.

8. The process as claimed in claim 1, wherein the said chiral amine in step (d) is selected from a group consisting of (S)-1-Phenylethylamine, (S)-1-bromophenylethylamine, (S)-1-methoxyphenylethylamine, (S)-1-tolylethylamine and (S)-(−)-1-(1-naphthyl)ethylamine, (R)-1-Phenylethylamine, (R)-1-bromophenylethylamine, (R)-1-methoxyphenylethylamine, (R)-1-tolylethylamine and (R)-(+)-1-(1-naphthyl)ethylamine.

9. The process as claimed in claim 1, wherein the solvent in step (d) is a solvent selected from water, toluene, t-butanol, xylene and acetonitrile, isopropyl acetate, dichloromethane, ethyl acetate, cyclohexane and mixtures thereof.

10. The process as claimed in claim 1, wherein the said intermediate 4 is produced in step (d) with 80-90% diastereomeric excess.

11. The process as claimed in claim 1, wherein the cyclizing agent in step (f) is selected from HCl, HBr, HI, HNO₃, CH₃COCl, SOCl₂, TMsCl, H₂SO₄ or any Lewis acid.

12. The process as claimed in claim 1, wherein the ring-opening agent in step (g) is selected from a group consisting of SOCl₂, ZnCl₂, acetic anhydride, acetic acid, LiOH, NaOH, KOH, HCl, HI and HBr.

13. The process as claimed in claim 1, wherein the amide in step (h) is selected from (S)-2-aminobutanamide, alkyl-(S)-2-aminobutanoate and salts thereof.

14. A chirally pure diastereomeric intermediate 5 synthesized by the process as claimed in claim 1:

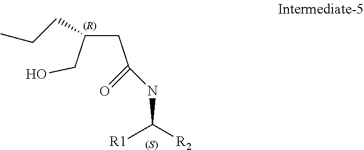

15. The diastereomeric intermediate 5 as claimed in claim 14 having a structure of:

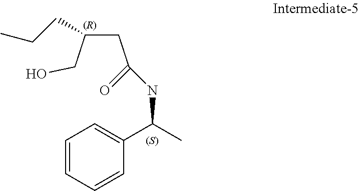

16. The diastereomeric intermediate 5 as claimed in claim 15 has 99.90-100% chiral purity.

17. The diastereomeric intermediate 5 as claimed in claim 15 has 75-85% yield.

18. A process for purifying diastereomeric Intermediate 4 forming Intermediate 5 comprising step of:
crystallizing the said Intermediate 4 with a mixture of solvents in a volume range of 5:95 to 40:60 producing 99.90-100% chirally pure Intermediate 5

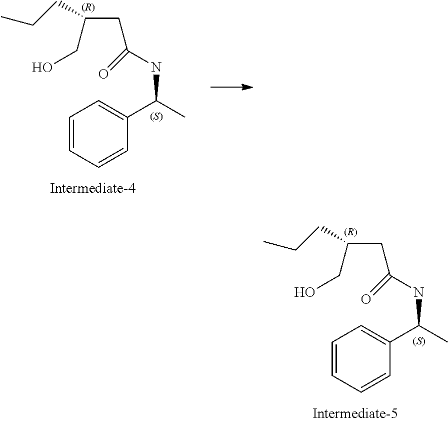

19. The process as claimed in claim 18, wherein the said mixture of solvents is selected from a group consisting of di-isopropyl ether, di-isopropyl acetate, diethyl ether, isopropyl acetate, methyl tertiary butyl ether and isopropyl acetate.

20. An enantiomerically pure Intermediate 6 with 99.90-100% enantiomeric excess synthesized by the process as claimed in claim 1:

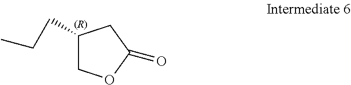

21. The enantiomerically pure Intermediate 6 as claimed in claim 20 has a yield of 90-95%.

22. A chirally pure Intermediate 11 having formula: 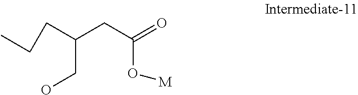
wherein, M is selected from a metal of Group I of the Periodic Table

23. The intermediate as claimed in claim 22, wherein M is Na or Li.

24. A process for preparing a chirally pure key Intermediate 11 as claimed in claim 22, wherein the said process comprises the step of reacting Intermediate 5 with a suitable base forming Intermediate 11: 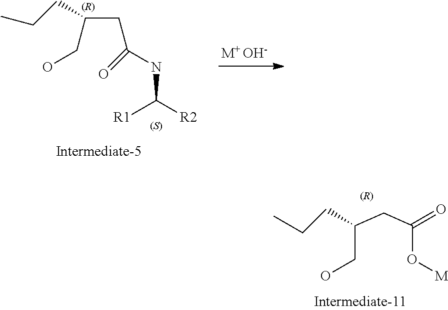
wherein R1 of Intermediate 5 is a substituted or unsubstituted aryl or heteroaryl; R2 of Intermediate 5 is a substituted or unsubstituted alkyl or cycloalkyl; and
M of Intermediate 11 is selected from a metal of Group I of the Periodic Table.

25. The process as claimed in claim 24, wherein the base is selected from LiOH, NaOH, KOH.
